(19) United States
(12) Patent Application Publication
(10) Pub. No.: US 2008/0039372 A1
(43) Pub. Date: Feb. 14, 2008

(54) Human chorionic gonadotropin antagonists and methods to prevent ovarian hyperstimulation

(75) Inventor: Joyce W. Lustbader, Tenafly, NJ (US)

Correspondence Address: WilmerHale/Columbia University, 399 PARK AVENUE, NEW YORK, NY, 10022, US

(73) Assignee: THE TRUSTEES OF COLUMBIA UNIVERSITY IN THE CITY OF NEW YORK, New York, NY (US)

(21) Appl. No.: 11/490,005

(22) Filed: Jul. 20, 2006

Related U.S. Application Data
(60) Provisional application No. 60/701,166, filed on Jul. 21, 2005.
(60) Provisional application No. 60/785,058, filed on Mar. 23, 2006.

(51) Int. Cl. A61K 38/24; A61P 15/00; A61P 35/00; C07K 14/59; C12N 1/00; C12P 21/00

(52) U.S. Cl. 514/8; 435/243; 435/69.1; 530/398

(57) ABSTRACT

The present invention is directed to a human chorionic gonadotropin (hCG) antagonist comprising deglycosylated hCG. Methods of recombinantly producing the hCG antagonist in a glycosylation-deficient host cell are also provided. Methods of treating hCG related conditions including ovarian hyperstimulation syndrome, gestational trophoblastic disease, and hCG related tumors by administering an effective amount of the hCG antagonist are also provided herein.

This application claims priority to U.S. Provisional Application No. 60/701,166 filed on Jul. 21, 2005 and U.S. Provisional Application No. 60/785,058 filed on Mar. 23, 2006, both of which are hereby incorporated by reference in their entireties.

This patent disclosure contains material, which is subject to copyright protection. The copyright owner has no objection to the facsimile reproduction by anyone of the patent document or the patent disclosure, as it appears in the U.S. Patent and Trademark Office patent file or records, but otherwise reserves any and all copyright rights.

Throughout this application, patent applications, published patent applications, issued and granted patents, texts, and literature references are cited. The disclosures of these publications in their entireties are hereby incorporated by reference into this application in order to more fully describe the state of the art as known to those skilled therein as of the date of the invention described and claimed herein.

The present invention was made with Government support under grant number awarded by National Institutes of Health with grant number NIDDK 5R01DK063224-02. Therefore, the U.S. Government may have certain rights in the invention.

### BACKGROUND OF THE INVENTION

Ovarian hyperstimulation syndrome (“OHSS”) is a potentially life-threatening complication of controlled ovarian hyperstimulation (“COH”) with exogenous gonadotropins. Approximately 1-2% of COH cycles are complicated by the severe form of OHSS, which is characterized by massive ovarian enlargement and extravasation of intravascular fluid into third space compartments. This vascular fluid leakage is thought to result from the overproduction of various ovarian vasoactive factors that increase capillary permeability (The Practice Committee of the American Society for Reproductive Medicine, Fertil Steril 80, 1309-1314 (2003)). In severe OHSS, the fluid shift is responsible for the development of ascites, pleural and/or pericardial effusions, hypovolemia, oliguria, and hemoconcentration. These processes may lead to life-threatening complications such as renal failure, adult respiratory distress syndrome, hemorrhage from ovarian rupture and thromboembolism.

Pharmacologic methods at triggering ovulation while reducing the risk of OHSS have been proposed for more than 20 years. Reducing the initial dose of delivered human chorionic gonadotropin (“hCG”) appears to lower the risk, although at the expense of decreasing the number of oocytes successfully recovered (Abdalla et al., Fertil Steril 48:958-963 (1987)). Recombinant human luteinizing hormone (“rhLH”) has been suggested with results suggesting a single dose of rhLH was effective in inducing follicular maturation and early luteinization comparable to 5000 International Units (“IU”) hCG (The European Recombinant Human Chorionic Gonadotrophin Study Group, Hum Reprod 15:1446-1451 (2000)). However, rhLH is not commercially available in the United States. Gonadotropin-releasing hormone (“GnRH”) agonists have also been used to trigger ovulation to provide a physiologic stimulation similar to endogenous LH surge (Hoff et al., J Clin Endocrionol Metab 57:892-896 (1983); Itskovitz et al., Fertil Steril 56:213-219 (1991)). Yet, it is unclear whether a sustained luteotropic effect and maintenance of multiple corpora lutea is possible given the shorter half-life of LH.

HCG is thought to play a crucial role in the development of OHSS. Several large studies have demonstrated that the incidence and severity of OHSS is greatest in cycles when hCG is administered to induce ovulation or provide luteal support, and in cycles with endogenous hCG production from a successful conception (Navot et al., Fertil Steril 58, 249-260 (1992)). Thus, one of the most effective methods of preventing OHSS is withholding hCG and avoiding pregnancy by cycle cancellation. Although it may be the safest approach, cycle cancellation is the least desirable alternative for the infertile patient, as it is both emotionally and financially burdensome.

Currently, there is no consensus for the best strategy to prevent OHSS. One approach is to cryopreserve all embryos after an in vitro fertilization (“IVF”) cycle in order to minimize hCG exposure without forfeiting oocyte retrieval (Amso et al., Fertil Steril 53, 1087-1090 (1990)). Studies have shown that the pregnancy potential of frozen embryos from hyperstimulated cycles is equal to or even greater to that of standard thawed embryo transfer (Federick et al., Fertil Steril 64, 987-990 (1995); Queenan et al., Hum Reprod 12, 1573-1576 (1997)). Yet in some of these studies, moderate to severe OHSS still occurred in a significant number of patients undergoing cryopreservation of all embryos (Queenan et al., Hum Reprod 12, 1573-1576 (1997); Salat-Baroux et al., Hum Reprod 5, 36-39 (1990)). It appears that the risk of development of OHSS remains unless all hCG is withheld from the cycle.

There is a need for a more effective treatment and/or prevention of OHSS. This and various other needs are addressed, at least in part, by one or more embodiments of the present invention.

### SUMMARY OF THE INVENTION

One aspect of the present invention is directed to a human gonadotropin (hCG) antagonist. The hCG antagonist of the present invention comprises a deglycosylated hCG. The deglycosylated hCG of the present invention comprises a reduced number of carbohydrate residues relative to naturally occurring hCG. In one embodiment, the deglycosylated hCG comprises less N-linked carbohydrates than naturally occurring hCG. In accordance with the present invention, the deglycosylated hCG may comprise a reduced number of sialic acid residues, N-acetylglucosamine residues, N-galactosamine residues, galactose residues, mannose residues, or fucose residues compared to naturally occurring hCG. The deglycosylated hCG may have at least about 50% to 90% less carbohydrates than naturally occurring hCG. In particular, the deglycosylated hCG may have at least about 55%, 60%, 65%, 70%, 75%, 80%, 85%, 90%, 95%, 97% or 99% less carbohydrates than naturally occurring hCG.

Another aspect of the present invention is further directed to a composition comprising the hCG antagonist and a pharmaceutically acceptable carrier. Suitable pharmaceutically acceptable carriers are known to those of skill in the art.

In one embodiment, the deglycosylated hCG of the present invention comprises at least one hCG-α subunit and at least one hCG-β subunit fused together. Additionally, these fused subunits may be subjected to neuraminidase or hydrogen fluoride treatment in order to produce a deglycosylated hCG. Alternatively, the deglycosylated hCG of the present invention may comprise at least one hCG-α subunit and at least one hCG-β subunit non-covalently bound together. In another embodiment, the deglycosylated hCG is produced by fusing the hCG-α and hCG-β subunits to produce a fused hCG and subjecting the fused hCG to neuraminidase. In another embodiment, the deglycosylated hCG is produced by fusing the hCG-α and hCG-β subunits to produce a fused hCG and subjecting the fused hCG to hydrogen fluoride treatment.

In addition, a method for making an hCG antagonist is provided. This method comprises transforming a host cell with a vector comprising a nucleic acid encoding hCG, wherein the host cell is glycosylation-deficient, and culturing the host cell under conditions sufficient to obtain expression of hCG, wherein the hCG is deglycosylated. In one embodiment, the host cell lacks a N-acetylglucosamine transferase enzyme. In another embodiment, the host cell is a Chinese hamster ovary cell. The host cell of the present invention may also be a bacteria, yeast, or insect cell. In one embodiment, the yeast cell is picchia. In another embodiment, the insect cell is baculovirus.

Another aspect of the present invention is further directed to a method for inhibiting the activity of naturally-occurring hCG via competitive inhibition. This method comprises administering an hCG antagonist comprising deglycosylated hCG in a hCG-antagonizing amount. This hCG antagonist may be administered orally, intravenously, intramuscularly, or subcutaneously. The present invention further provides that the hCG antagonist may be administered in combination with hormones or chemotherapeutic agents. Suitable hormones for the present invention include gonadotropins (with or without a GnRH antagonist), and post ovulation progesterone. In accordance with the present invention, the chemotherapeutic agent can be, for example, bleomycin, daunomycin, 5-FU, cytosine arabinoside, colchicine, cytochalasin B, daunorubicin, neocarcinostatin, suramin, doxorubicin, carboplatin, taxol, mitomycin C, vincristine, vinblastine, methotrexate, and cisplatin. Further in accordance with the present invention, the hCG antagonist can be administered in an amount ranging from about 3,000 IU to about 10,000 IU. In another embodiment, the amount of hCG antagonist that can be administered is from about 2,500 IU to about 9,500 IU; from about 3,500 IU to about 9,000 IU; from about 4,000 IU to about 8,500 IU; from about 4,500 IU to about 8,000 IU; from about 5,000 IU to about 7,500 IU; from about 5,500 IU to about 7,000 IU; from about 6,000 IU to about 6,500 IU, or other ranges within the general range of 2,000 IU to about 12,000 IU.

In another embodiment, the subject receiving the hCG antagonist suffers from ovarian hyperstimulation syndrome, gestational trophoblastic disease, proliferation of circulating hCG, proliferation of hCG-α, proliferation of hCG-β, tumors secreting hCG, and tumors responsive to hCG or cells with hCG receptors. Also according to the present invention, a method for treating OHSS in a subject is provided comprising administering to a subject an effective amount of the hCG antagonist comprising deglycosylated hCG. In another embodiment, a method of preventing OHSS in a subject is provided comprising administering to the subject a hCG antagonist comprising deglcosylated hCG in a hCG-antagonizing amount. In another embodiment, the subject is an egg donor. In another embodiment, the subject suffering from ectopic pregnancy. In another embodiment, the hCG antagonist is administered as a contraceptive agent to prevent pregnancy.

In accordance with the present invention, a method for treating a subject suffering from gestational trophoblastic disease from increased circulation of hCG is provided in which an effective amount of a hCG antagonist comprising deglycosylated hCG is delivered to a subject in need thereof. The hCG antagonist may act by inhibiting the binding of the proliferating hCG to the hCG receptor.

The present invention further provides a method for treating a subject suffering from a hCG-associated tumor. This method comprises administering to the subject an effective amount of a hCG antagonist comprising deglycosylated hCG. The hCG-associated tumor may be of placental or germ cell origin. In one embodiment, the hCG-associate tumor may be a relapsing choriocarcinoma or a testicular germ cell tumor. In another embodiment, the patient may be suffering from testicular cancer, biliary cancer, pancreatic cancer, lung carcinoma, or bladder carcinoma. According to the present invention, the hCG-associated tumor may be stimulated by hCG or may secrete hCG and proliferate in a feedback mechanism stimulated by the circulating hCG. In one embodiment, the hCG-associated tumor is treated wherein the hCG antagonist acts by inhibiting the binding of naturally occurring hCG to the hCG receptor via competitive inhibition.

The present invention further provides a method of inhibiting growth of human chorionic gonadotropin (hCG)-associated tumor cells. This method comprises administering to a subject in need thereof an effective amount of a human chorionic gonadotropin (hCG) comprising deglycosylated hCG. The hCG-associate tumor cell can be, for example, a lung carcinoma tumor cell or a bladder carcinoma tumor cell. The hCG antagonist may inhibit tumor cell growth by inhibiting the binding of naturally occurring hCG to the hCG receptor via competitive inhibition.

The invention provides an hCG having one, two, or three of the following N-linked glycosylation mutated, so as to reduce glycosylation of the hCG: asparagine at position 13, asparagine at position 30, or asparagine at position 52.

The invention also provides a human chorionic gonadotropin antagonist comprising (i) a polypeptide having an amino acid sequence of an α subunit of hCG (SEQ ID NO:2) except that the asparagine at position 52 is mutated; and (ii) a polypeptide having an amino acid sequence of β subunit of hCG (SEQ ID NO:3) except that the asparagine at position 13 and the asparagine at position 30 are mutated. In one embodiment, the α and β subunits are fused together. In another embodiment, fusion is via a peptide linker.

### BRIEF DESCRIPTION OF THE FIGURES

FIGS. 1A and 1B: FIG. 1A depicts the effect of β-hCG on cell proliferation. FIG. 1B depicts the effect of α-hCG on cell proliferation as described in Example 1.

FIG. 2: ¹²⁵I-β-hCG binding to neoplastic cells as described in Example 2.

FIG. 3: hCG binding to human endometrial and granulose cells in vitro as described in Example 3.

FIG. 4: Results of a competitive binding assay of hCG to the receptor.

FIG. 5: Results of a cAMP assay.

FIG. 6: Ultrasound of multifollicular, enlarged ovary and pelvic ascites associated with OHSS in humans.

FIG. 7: Comparison of the primary amino acid and carbohydrate structures of the glycoprotein hormones. The increased half-life of hCG is attributed to its unique O-linked carbohydrate moities.

FIG. 8: Diagram depicting the yoked hCG-antagonist. Site-directed mutagenesis was used to develop the yoked hCG-antagonist with removal of three of the four N-linked glycosylation sites (Asn52, Asn13 and Asn30) to prevent LH receptor activation. One site, Asn78, on the alpha subunit was left intact to enhance receptor binding. The effect is that the hCG-antagonist binds but does not activate the receptor.

FIG. 9: Assessment of in vitro binding of the hCG-antagonist by a competitive binding assay in Chinese hamster ovary cells transfected with the LH receptor (CHO-LH).

FIG. 10: Assessment of in vitro bioactivity of the hCG-antagonist by measuring cAMP activity in CHO-LH cells. While hCG increased intracellular cAMP, hCG-antagonist was unable to stimulate cAMP levels above baseline.

FIG. 11: Ovaries from PMSG/hCG superovulated rats (left panel) contain more ovarian stigma, or remnants of ovulated oocytes, than those treated with PMSG/hCG-Ant (right panel). Also see Table 1.

FIG. 12: Femoral Vein injections: Dissection of rat femoral triangle and injection of Evan's blue dye into femoral vein for vascular permeability studies.

FIG. 13: Vascular permeability slide: The upper panel shows ovaries and uterus from PMSG/hCG group. The lower panel shows ovaries from PMSG/hCG-Ant group. Rats received 4 days of PMSG followed by HCG and vascular permeability studies using Evan's blue dye were performed at 48 hrs. The PMSG/hCG ovaries and uterus retained significantly more dye than those that received hCG-ant, suggesting decreased vascular permeability in animals receiving hCG-ant.

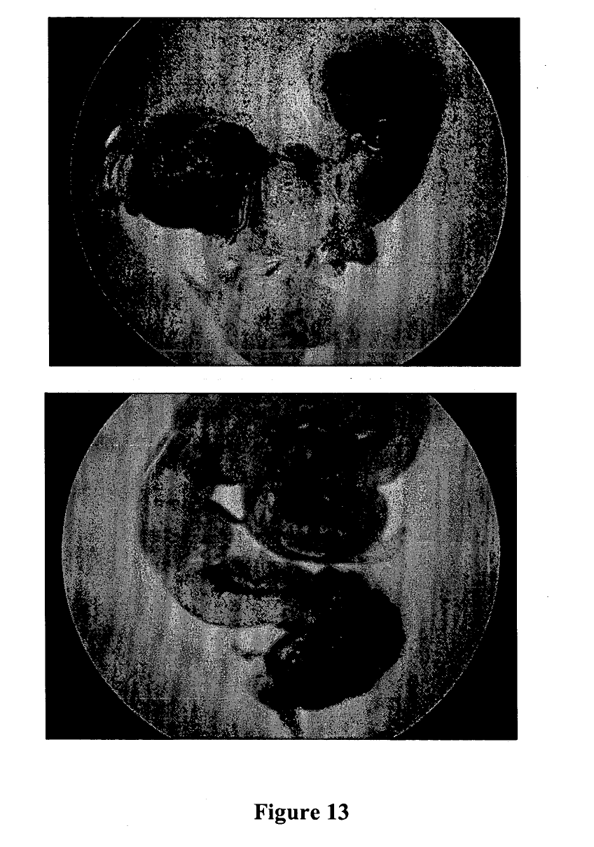

FIG. 14: Rats treated with hCG-antagonist had 85% less dye extravasation into the peritoneal fluid compared to hCG. Rats treated with hCG followed by hCG-antagonist had a >99% decrease in dye extravasation compared to hCG.

FIG. 15: The nucleic acid sequence of the yoked hCG βN(13,30)K+αN(52)K is shown as SEQ ID NO:1 (the mutated codons are underlined).

### DETAILED DESCRIPTION OF THE INVENTION

For the purposes of promoting an understanding of the principles of the invention, reference will now be made to embodiments and specific language will be used to describe the same. It will nevertheless be understood that no limitation of the scope of the invention is thereby intended, such alteration and further modifications of the invention, and such further applications of the principles of the invention as illustrated herein, being contemplated as would normally occur to one skilled in the art to which the invention relates.

The present invention is directed to a human gonadotropin (“hCG”) antagonist comprising deglycosylated hCG. hCG is a placental hormone that maintains the steroid secretions of the corpus luteum during early pregnancy. It is a member of a family of glycoprotein hormones that are dimers formed from the noncovalent association of α subunits common to all members of the family and distinctive β subunits that designate the target specificity of the different hormones. Both subunits are heavily glycosylated. The 92-residue α-chain (GenBank Accession No. 1HRPA; also shown as SEQ ID NO:2) contains two sites of N-linked glycosylation, and the 145 residue β-chain (GenBank Accession No. 1HRPB; also shown as SEQ ID NO:3) has two N-linked glycosylation sites and four O-linked glycosylation sites on its unique carboxy-terminal extension (Pierce et al., Annu Rev Biochem 50, 465-495 (1981)). The O-linked glycosylation sites are not involved in the bioactivity of hCG. (Matzuk et al. “The biological role of the carboxyl-terminal extension of human chorionic gonadotropin beta-subunit.” Endocrinology, 1990 January; 126(1):376-83.) Carbohydrate content in native hCG accounts for 30 to 35% of its 38,900 Da of molecular mass (Kessler et al., J. Biol. Chem. 254, 7901-7908 (1979)).

“Deglycosylated hCG” is one class of hCG antagonists and as used herein refers to hCG which has a reduced number of carbohydrate residues relative to naturally occurring hCG. Deglycosylated hCG binds strongly to its receptor, but adenylate cyclase activation and steroidgenesis are greatly impaired unless the α-chain is glycosylated at its first N-linked site (Matzuk et al., J Biol Chem 264, 2409-2414 (1989)). However, hCG deglycosylated by neuraminidase treatment retains significant biological activity (Moyle et al., J Biol Chem 250, 9163 (1975)), while hydrogen fluoride (“HF”) treated hCG binds to the receptor but lacks biological efficacy (Chen et al., J Biol Chem 257, 1444 (1982)). It was found that HF-hCG could not activate cAMP or testosterone production in intact rat Leydig cells. Cyclic AMP activity assays are used to measure the biological activity of hCG. However, HF-hCG failed to inhibit the uterotrophic action of hCG in immature female rats (Chen et al., J Bio Chem 257, 1444 (1982)). Thus, a pure hCG antagonist effective in both male and female in vivo models has yet to be engineered from deglycosylated hCG.

The deglycosylated hCG of the present invention has a reduced number of carbohydrate residues relative to naturally occurring hCG. In one embodiment, the deglycosylated hCG of the present invention comprises a reduced number of N-linked carbohydrates relative to naturally occurring hCG. In one embodiment, the deglycosylated hCG has its alpha and beta subunits ligated together (fused, or yoked) and has had three of the four N-linked carbohydrate sites altered by site specific mutagenesis. The N-linked carbohydrates that may be reduced in the deglycosylated hCG of the present invention include sialic acid residues, N-acetylglucosamine residues, N-galactosamine residues, galactose residues, mannose residues, and fucose residues. The deglycosylated hCG of the present invention may have at least 50% to 90% less carbohydrates than naturally occurring hCG and has about 75% less carbohydrates than naturally occurring hCG. In particular, the deglycosylated hCG may have at least 55%, 60%, 65%, 70%, 75%, 80%, 85%, 90%, 95%, 97% or 99% less carbohydrates than naturally occurring hCG. This deglycosylated hCG can then be used as an effective hCG antagonist in vivo.

Neuraminidase treatment of hCG is not ideal because such hCG retains some residual biological activity. The deglycosylated hCG of the present invention may be recombinantly produced in any host cell that is glycosylation deficient. “Glycosylation deficient” as used herein is defined as lacking the glycosylation machinery and/or ability to properly glycosylate hCG. For example, the glycosylation deficient host cell may lack a N-acetylglucosaminyl transferase enzyme. It will be appreciated by those of skill in the art that any suitable glycosylation deficient host cell may be used in the present invention. Non-limiting examples include Chinese hamster ovary (“CHO”) cells, bacterial cells, yeast cells (i.e., picchia), and insect cells (i.e., baculovirus). In one embodiment, the recombinant hCG of the present invention is produced by a Chinese hamster ovary (CHO) cell lacking the appropriate glycosylation machinery. For example, Lec 1 is a CHO cell line that is deficient in the enzyme N-acetylglucosaminyltransferase I (GlcNAc-TI) (Stanley et al., Cell 6, 121-128 (1975); Stanley, P., Glycobiology 2, 99-107 (1992)); therefore, it creates deglycosylated hCG. Lec1 cells are unable to synthesize complex or hybrid-type N-linked glycans as they are unable to initiate the conversion of oligomannosyl to complex N-linked glycans. The lack of GlcNAc-TI in the Lec1 mutant is not lethal to CHO cells and enables researchers to use Lec1 cells to produce recombinant glycoproteins with truncated, simple oligomannosyl N-glycans (Stanley, P., Glycobiology 2, 99-107 (1992); Butters et al., Protein Sci. 8, 1696-1701 (1999)). Therefore, according to the present invention, the Lec1 cell line may be used to produce the deglycosylated hCG.

The deglycosylated hCG of the present invention may be expressed by the host cell using methods known to those of ordinary skill in the art. For instance, the host cell may be transformed with a vector comprising a nucleic acid encoding hCG. hCG is a known protein and the nucleic acid molecules encoding hCG have been previously disclosed, for example, in Fiddes and Goodman, 1980, “The cDNA for the beta-subunit of human chorionic gonadotropin suggests evolution of a gene by readthrough into the 3′-untranslated region.”; Nature 286(5774):684-7. However, the present invention is not limited herein to the hCG protein and nucleic acid disclosed previously. One having skill in the art would be capable of selecting a suitable nucleic acid encoding hCG for use in the present invention. As used herein, the “nucleic acid,” “nucleic acid molecule,” or “polynucleotide” refers to a single- or double-stranded DNA, genomic DNA, cDNA, RNA, DNA-RNA hybrid, or nucleotide polymer. The nucleic acid encoding the hCG protein provided by this invention can be assembled from cDNA fragments and short oligonucleotide linkers, or from a series of oligonucleotides, to provide a synthetic gene which is capable of being inserted in a recombinant expression vector and expressed in a recombinant transcriptional unit.

In one embodiment of the invention, the hCG nucleic acid is present in a suitable expression vector. The term “expression vector” refers to a plasmid, virus or other vehicle known in the art that has been manipulated by insertion or incorporation of the hCG nucleic acid. Polynucleotide sequences, which encode hCG, may be operatively linked to expression control sequences. “Operatively linked” refers to a juxtaposition wherein the components so described are in a relationship permitting them to function in their intended manner. An expression control sequence operatively linked to a coding sequence is ligated such that expression of the coding sequence is achieved under conditions compatible with the expression control sequences. As used herein, the term “expression control sequences” refers to nucleic acid sequences that regulate the expression of a nucleic acid sequence to which it is operatively linked. Expression control sequences are operatively linked to a nucleic acid sequence when the expression control sequences control and regulate the transcription and, as appropriate, translation of the nucleic acid sequence. Thus, expression control sequences can include appropriate promoters, enhancers, transcription terminators, a start codon (i.e., ATG) in front of a protein-encoding gene, splicing signal for introns, and maintenance of the correct reading frame of that gene to permit proper translation of mRNA, and stop codons. The term “control sequences” is intended to include, at a minimum, components whose presence can influence expression, and can also include additional components whose presence is advantageous, for example, leader sequences and fusion partner sequences. Expression control sequences can include a promoter.

By “promoter” is meant the minimal sequence sufficient to direct transcription. Also included in the invention are those promoter elements which are sufficient to render promoter-dependent gene expression controllable for cell-type specific, tissue-specific, or inducible by external signals or agents; such elements may be located in the 5′ or 3′ regions of the gene. Both constitutive and inducible promoters are included in the invention (see e.g., Bitter et al., Methods in Enzymology 153:516-544 (1987)). When cloning in mammalian cell systems, promoters derived from the genome of mammalian cells (e.g., metallothionein or elongation factor-1 alpha promoter) or from mammalian viruses (e.g., the retrovirus long terminal repeat; the adenovirus late promoter; the vaccinia virus 7.5K promoter; the cytomegalovirus promoter; the Rous Sarcoma virus promoter; the Moloney Sarcoma virus promoter) may be used. Promoters produced by recombinant DNA or synthetic techniques may also be used to provide for transcription of the nucleic acid sequences of the invention.

Moreover, useful expression vectors can further comprise a selectable marker that may be used to ascertain successful incorporation of the intended nucleic acid. Suitable selectable markers include green fluorescent protein, antibiotic resistance, such as for ampicillin and tetracycline resistance, neomycin, zeocin, hygromycin, and recessive markers such as thymidine kinase (TK), dihydrofolate reductase (DHFR), adenine phosphoribosyl transferase (APRT) and hypoxanthine phosphoribosyl transferase; thus providing a simple means for identifying transformed cells.

As used herein, a host cell is transformed with the hCG nucleic acid by introducing or inserting the nucleic acid into the host cell. “Introducing” the nucleic acid encompasses any method of inserting an exogenous nucleic acid molecule into a cell and includes, but is not limited to, transduction, transfections, microinjection, and viral infection of the host cells. Ideally, the choice of a nucleic acid delivery system will be made by those of skill in the art.

The expression of hCG in the transformed cell may be limited in time or transient. “Transient expression” indicates that the transformed cell expresses the gene product encoded by the inserted nucleic acid for short periods of time or only during proliferation and expansion of the cells.

The deglycosylated hCG of the present invention may also be made by other conventional methods known to those of skill in the art. For instance, the deglycosylated hCG of the present invention may be made by treating hCG with hydrogen fluoride (“HF”) as described in Chen et al., J Biol Chem 257, 1444 (1982). The deglycosylated hCG of the present invention may also be made by neuraminidase treatment as described in Moyle et al., J Biol Chem 250, 9163 (1975).

Furthermore, the deglycosylated hCG of the present invention may be made by fusing at least one hCG-α subunit and at least one hCG-β subunit together (this fusion product is also known an “yoked” hCG). It may be advantageous to fuse the hCG α and β subunits as these subunits normally come apart during purification because they are not covalently bonded. In one embodiment, the hCG α and β subunits may be fused by gene splicing, thereby joining their coding regions into a co-linear sequence. The hCG with the fused α and β subunits can then be treated with neuramindase or HF in order to provide a deglycosylated hCG. Alternatively, the α and β subunits of the deglycosylated hCG of the present invention may be non-covalently bound together.

According to the present invention, one or both of the hCG α and β subunits may comprise less carbohydrates than naturally occurring hCG. For instance, the deglycosylated hCG of the present invention may have at least one α subunit that has less carbohydrates than a naturally occurring hCG-α subunit; for example, at residues 52 and 78. The deglycosylated hCG of the present invention may alternatively (or, in addition) have at least one β subunit that has less carbohydrates than a naturally occurring hCG-β subunit; for example, at residues 13 and 30.

As described above, the deglycosylated hCG of the present invention binds to the hCG receptor but lacks biological activity due to its deglycosylation. (Matzuk et al., J Biol Chem 264, 2409-2414, (1989)). Because the deglycosylated hCG binds to the hCG receptor, it acts as a hCG antagonist. “hCG antagonist” as used herein is defined as a molecule that binds to the hCG receptor at a specific (active) site on that protein. This binding suppresses or inhibits the activity and/or function of the hCG receptor protein. The hCG antagonist of the present invention also inhibits the activity of naturally-occurring hCG via competitive inhibition. “Competitive inhibition” as used herein is defined as blockage of the action of an enzyme on its substrate by replacement of the substrate with a similar but inactive compound that can combine with the active site of the enzyme but that is not acted upon or split by the enzyme. Therefore, the present invention is also directed to a method of inhibiting the activity of naturally-occurring hCG via competitive inhibition comprising administering an hCG antagonist comprising deglycosylated hCG in a hCG-antagonizing amount. A “hCG-antagonizing amount” as used herein is defined as an amount effective to inhibit the activity and/or function of the hCG receptor.

The deglycosylated hCG of the present invention may be administered to a patient in need thereof. The hCG antagonist may be administered in any manner known to one of ordinary skill in the art, for example, orally, intravenously, intramuscularly, or subcutaneously. The deglycosylated hCG of the present invention may be administered in an amount ranging from about 3,000 IU to 10,000 IU. In another embodiment, the amount of hCG antagonist that can be administered is from about 2,500 IU to about 9,500 IU; from about 3,500 IU to about 9,000 IU; from about 4,000 IU to about 8,500 IU; from about 4,500 IU to about 8,000 IU; from about 5,000 IU to about 7,500 IU; from about 5,500 IU to about 7,000 IU; from about 6,000 IU to about 6,500 IU, or other ranges within the general range of 2,000 IU to about 12,000 IU. According to the present invention, the deglycosylated hCG may also be administered in combination with other drugs and/or hormones. For instance, deglycosylated hCG may be administered to a patient both before and after the delivery of many different hormone preparations. In this regard, hormones contemplated for use in the method of the invention include gonadotropins to stimulate the ovaries (with or without a gonadotropin-releasing hormone (“GnRH”) antagonist), and post ovulation progesterone. In addition, the deglycosylated hCG of the present invention may also be administered along with one or more chemotherapeutic agents. “Chemotherapeutic agents” as used herein are defined as agents having an antitumor or cytotoxic effect. Such agents include bleomycin, daunomycin, 5-FU, cytosine arabinoside, colchicine, cytochalasin B, daunorubicin, neocarcinostatin, suramin, doxorubicin, carboplatin, taxol, mitomycin C, vincristine, vinblastine, methotrexate, and cisplatin. Other chemotherapeutic agents will be known to those of skill in the art (see for example The Merck Index).

The methods of the present invention may be used to treat a patient suffering from any condition in which hCG plays a role. For instance, the deglycosylated hCG of the present invention may be used to treat a subject suffering from ovarian hyperstimulation syndrome, gestational trophoblastic disease, proliferation of circulating hCG, proliferation of hCG-β, proliferation of hCG-α, tumors secreting hCG, or tumors responsive to hCG or to cells with hCG receptors (either directly or via a feedback mechanism stimulated from circulating hCG). Therefore, the methods of the present invention may be used to treat a subject suffering from a hCG-associated tumor. A “hCG-associated tumor” as used herein is defined as a tumor cell that expresses and/or requires hCG or hCG subunits for growth HCG-associated tumors/cancers contemplated include, but are not limited to, trophoblastic tumors of placental and germ cell origin, relapsing choriocarcinomas, testicular germ cell tumors, testicular cancer, biliary cancer, pancreatic cancer, lung carcinoma, and bladder carcinoma. For instance, patients with gestational trophoblastic disease, including benign mole, could benefit by the methods of the present invention since many of their symptoms, including corpus luteum (“CL”) cysts, are believed to be related to their very high levels of circulating hCG.

With regard to the tumors treated by the present invention, there is data indicating that hCG and its subunits proliferate cell growth on a number of different cancer cell lines. Furthermore, hCG presents in a number of different pathologies other than pregnancy. Indeed, the presence of hCG in non-pregnant physiological conditions suggests additional functions of hCG. HCG is synthesized as individual subunits and is assembled into a heterodimer prior to secretion. A physiological role has only been elucidated for the heterodimer, while the biological significance of free subunits, as well as β-core hCG, has yet to be determined. There is a large body of data that demonstrates the presence of the free subunits and β-core hCG in disease states such as ectopic pregnancy (Cole et al., J Clin Endocrinol Metab 78:497-499, (1994)), trophoblastic disease (Cole et al., J Reprod Med 39:193-200, (1994)), and a number of malignant neoplasms (Marcillac et al., Cancer Research 52:3901-3907, (1992)). These observations suggest an alternative role for hCG with possible paracrine/autocrine activities. Since hCG or its subunits are secreted by a number of cancers, the hCG antagonist of the present invention can be used to inhibit the LH/CG receptor, thereby inhibiting cell growth in these proliferating cells. The modified hCG described herein binds to the LH receptor but is unable to activate the receptor. Asparagine 52 on the alpha subunit is required for bioactivity. Therefore, the invention encompasses hCG antagonists that comprise hCG with the asparagines 52 residue on the alpha subunit ablated, substituted, deleted or otherwise deemed non-functional so as to inhibit bioactivity of hCG.

To summarize evidence suggesting an alternate role for hCG in vivo includes: 1) the abundant presence of free subunits in normal pregnancies and patients with severe neoplastic diseases (Marcillac et al., Cancer Research 52:3901-3907 (1992)), 2) mitogenic activity by free subunits of hCG, 3) the presence of hCG receptors on non-gonadal tissues such as the endometrium, myometrium and fallopian tubes (Kornyei et al., Biology of Reproduction 49:1149-1157 (1993); Lincoln et al. J Clin Endocrinol Metab 75:1140-1144 (1992); Lei et al., J Clin Endocrinol Metab 77:863-872 1(1993)), 4) the production of hCG by the embryo at the early blastocyst stage during the preimplantation period (Hay et al., J Clin Endocrinol Metab 67:1322-1324 (1998); Dimitriadou et al., Fertility & Sterility 57:631-636 (1992)), and 5) the present inventor's crystallography studies demonstrating a compelling structural relationship between hCG and many growth factors. Also, some neoplasms and cell lines secrete α-subunit and β-subunit (Iles et al., British Journal of Cancer 61:663-666 (1990)) as putative autocrine factors and, in this regard, it has been shown that the addition of anti-α-subunit antibodies can inhibit the growth of lung carcinoma cells in vivo and in vitro (Kumar et al., J Natl Cancer Inst 84:42-47 (1992)). Therefore, existing data supports a likely autocrine/paracrine role for hCG and its subunits.

Therefore, hCG and its subunits may be growth factors or inducers of cell differentiation. HCG can promote differentiation of cytotrophoblasts into syncytiotrophoblasts (Shi et al., Endocrinology 132:1387-1395 (1993)), and free α-subunit stimulates prolactin production by the decidua (Blithe et al., Endocrinology 129:2257-2259 (1991)). The production of hCG by the early blastocyst (Dokras et al., Human Reproduction 6:1143-1151 (1991); Woodward et al., Human Reproduction 9:1909-1914 (1994); Dokras et al., Human Reproduction 8:2119-2127 (1993); Dokras et al., Human Reproduction 6:1453-1459 (1991); Lopata et al., Human Reproduction 4:87-94 (1989)) suggests that hCG promotes the viability of the embryo and facilitates implantation. The presence of receptors for hCG on the endometrial surface (Lincoln et al., J Clin Endocrinol Metab 75:1140-1144 (1992); Bonnamy et al., Endocrinology 132:1240-1246 (1993)) suggests that it supports implantation by reprogramming endometrial cells, blocking apotosis (Tapanainen et al., Mol Endocrinol 7:643-650 (1993)), which normally follows the regular menstrual cycle, so the secretory phase may continue (Gompel et al., Am J Pathol 144:1195-1202 (1994)).

It is thought that neither the α- or β-subunit individually bind the LH/CG receptor, and that both subunits are required for activity (Puett et al., Mol Cell Endocrinol 125:55-64 (1996)). Nonetheless, a β-homodimer of hCG binds the receptor with a relatively high affinity is biologically active (Lobel et al., Endocrine 10:261-270 (1999)). The α-homodimer induces cAMP activity. Unique combinations of the various loops of the α- and β-subunits have an affinity for the hLH/CG receptor and can induce cAMP production (Moyle et al., Chemistry & Biology 5:241-254 (1998)). Therefore, there is a body of evidence that suggests that the individual subunits themselves may play a role in activating the hLH/CG receptor in either a physiologic state or a disease state where ectopic production of a large amount of a single subunit has been identified.

Given the role of hCG in neoplastic growth, the deglycosylated hCG of the present invention may be used for competitive binding to a variety of cell lines that express and/or require hCG subunits for growth. The deglycosylated hCG of the present invention may, therefore, be used to inhibit growth of these neoplastic cells that express and/or require active hCG subunits for growth. For example, the hCG antagonist of the present invention may inhibit the growth of lung or bladder cancer cells by binding to hCG receptors, thereby inhibiting the activity of naturally occurring hCG due to competitive inhibition. In one embodiment of the invention, the hCG antagonist can be an individual subunit of hCG modified so that it functions as an antagonist, such as competitive inhibitor, where it binds to the receptor, but does not result in activation of the receptor.

Therefore, the present invention provides methods for treating hCG-related conditions in a subject comprising administering to the subject an effective amount of the hCG antagonist comprising deglycosylated hCG. “Effective amount” as used herein is defined as the amount of the hCG antagonist effective to bind the hCG receptor, thereby inhibiting hCG activity by competitive inhibition. For example, in one embodiment, the present invention provides for a method of treating OHSS in a subject comprising administering an effective amount of hCG antagonist comprising deglycosylated hCG. In another embodiment, the present invention provides a method for preventing OHSS in a subject comprising administering to the subject in need thereof a hCG antagonist comprising deglycosylated hCG in a hCG-antagonizing amount. According to the present invention, the subject receiving the deglycosylated hCG may also be receiving hCG therapy. In one embodiment of the invention, a subject undergoing HCG therapy could also receive deglycosylated hCG to prevent OHSS. Both hCG and the deglycosylated hCG would be given in the range of 3,000 IU to 10,000 IU, the exact combination dosage would be specific to the individual patient's unique physiological requirements.

An hCG antagonist could be a useful adjunct to prevent OHSS when cryopreserving all embryos. Theoretically, it could be given immediately after oocyte retrieval to cease hCG-driven production of ovarian vasoactive substances, and hence prevent the development of severe OHSS. In addition, an effective hCG antagonist could be administered to all egg donor patients after oocyte retrieval, not only to prevent OHSS, but also to block corpus luteum stimulation, minimize ovarian stimulation symptoms, and hasten the onset of menses after the completion of the cycle. Decreasing the discomfort and risks inherent to ovarian stimulation would make the procedure more tolerable, perhaps increasing patient compliance and participation in egg donor programs.

The hCG antagonist of the present invention may also be combined with a pharmaceutically acceptable carrier. Such carriers are known in the art and include sterile aqueous or non-aqueous solutions, suspensions and emulsions. Examples of non-aqueous solvents include propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, fixed oils, and the like. Vehicles suitable for intercellular or intracellular injection may also include fluid and nutrient replenishers, electrolyte replenishers, such as those based on Ringer's dextrose, for example. Preservatives and other additives may also be present. For example, antimicrobials, antioxidants, chelating agents, and inert gases may also be used.

Reference will now be made to specific examples illustrating the compositions and methods above. It is to be understood that the examples are provided to illustrate embodiments and that no limitation to the scope of the invention is intended thereby.

### EXAMPLES

### Example 1

### Growth Activity of hCG Free α and β Subunits on Neoplastic Cell Cultures In Vitro

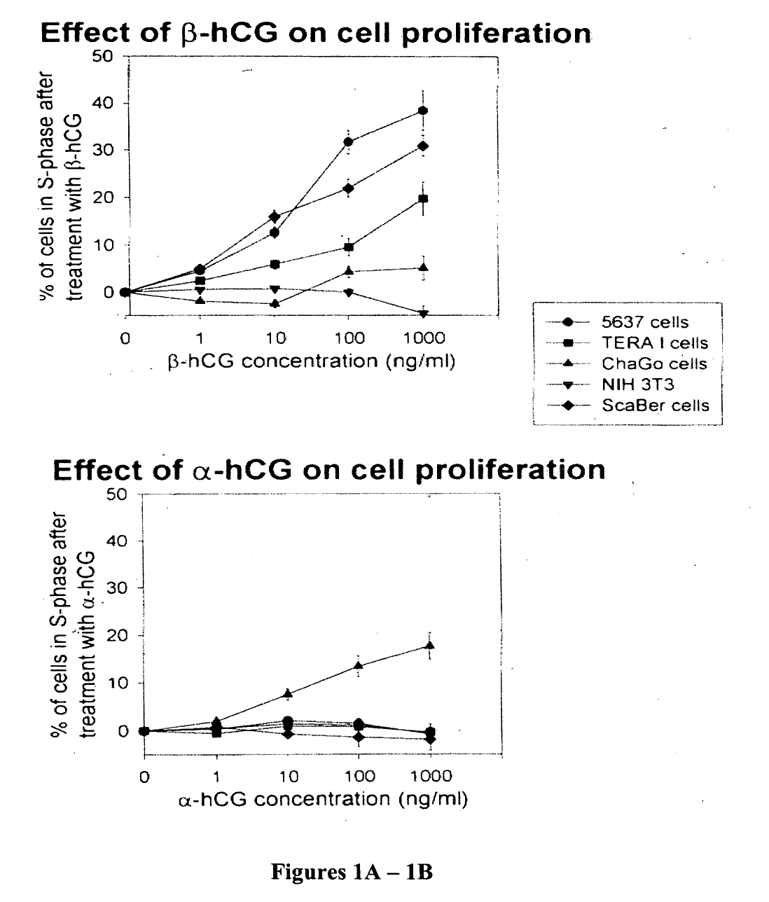

To test the stimulatory effect of free subunits on cell growth and proliferation several cell lines were used (all purchased from American Cell Type Collection) and cultured in the presence and absence of hCG or its free subunits. SCaBER, human bladder carcinoma, used in these studies is known to secrete high levels of free β-hCG (Iles et al., British Journal of Cancer 61:663-666 (1990)), which acts as an autocrine growth promoter. Cell line 5637, which is also a human bladder carcinoma, (an intermediate producer of β-hCG) (Iles et al. (1990)), and the germ cell tumor line TERA I, which does not secrete any detectable amounts of β-hCG ((Iles et al. (1990)), were also used in these studies. In addition to β-hCG producing cell lines, ChaGo-K-1 cells, human lung carcinoma, which are producers of high levels of free α-hCG (Kumar et al., J Natl Cancer Inst 84:42-47 (1992)), were also used. Cells were seeded in DMEM media, containing 10% fetal calf serum, antibiotics and glutamine. To exclude the effect of endogenous free subunits on cell proliferation, the media which contained no hCG-related molecules was changed every 6 hours during the 2-day period. In the last two changes the media was supplemented with different concentrations of hCG or free subunits. After a 12 hour incubation (2×6 h) in media, containing exogenous hCG or free subunits, cells were washed with Hanks solution and collected by trypsin-EDTA treatment. Following 3 washes, cells were fixed with 70% ethanol and treated with RNase as previously described (Gray et al., Int J Radiat Biol Relat Stud Phys Chem Med 49:237-255 (1986)). The final cell preparation was treated with propidium iodide and analyzed using flow cytometry. A marked increase was found in the fraction of cells in S-phase for free β-hCG treated SCaBER and 5637 cell lines (See FIG. 1A), while a moderate effect on TERA I cells was found and no effect on ChaGo-K-1 cells was found. For free α-subunit, the growth response was found only on the ChaGo-K-1 cells (See FIG. 1B), which corresponds to published reports by Talwar et al., J Natl Cancer Inst 84:42-47 (1992), on the dependence of these cells for free α-subunit. These experiments demonstrate that free hCG subunits and hCG play a role in cell growth and proliferation.

### Example 2

### Binding of hCG Free α and β Subunits to Cells Responding to β-hCG

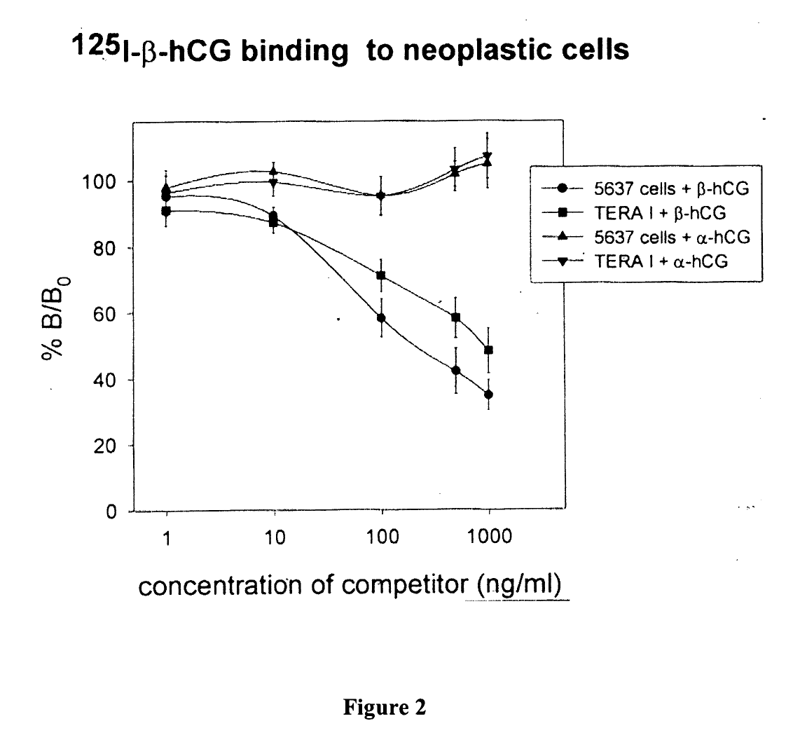

To identify free subunit receptors, hCG, β-hCG and α-hCG were labeled with ¹²⁵I and binding assay was performed for these ligands on whole cells in suspension. In this experiment the SCaBER, 5637 and TERA I cell lines were tested. Prior to binding, cells were grown to a subconfluent state (⅔ of the 60 mm tissue culture dish was covered), removed by EDTA solution, washed 3 times in Hanks solution followed by the addition of labeled ligand (150-180×10³ cpM/10⁶ cells) in the presence of increasing amounts of ‘cold’ ligand. The cells were incubated 1 h at 37° C. and then 4 hours at 4° C. Then cells were spun, the supernatant aspirated and the cell pellet was counted on a gamma-counter. NIH 3T3 cells and mouse myeloma X63.Ag8.653 were used as controls for non-specific binding. The results are presented in FIG. 2. No binding of hCG dimer or free α-subunit to any of these cells was found. On the other hand, 5637 cells (intermediate producer of β-hCG) and TERA I (non-producer) bound ¹²⁵I-β-hCG. The specificity of this binding was confirmed by dose dependent competition with ‘cold’ β-hCG (See FIG. 2).

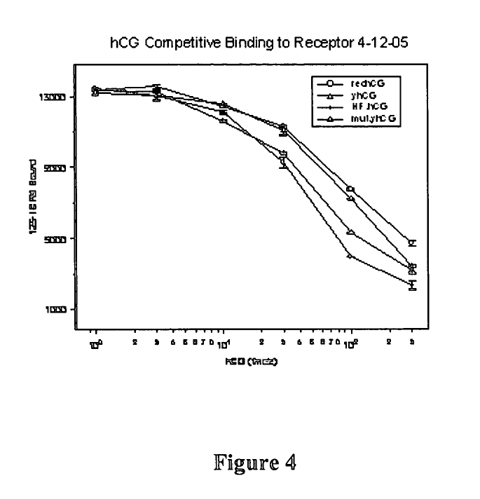

The binding assay of hCG competitive binding to receptor (FIG. 4) shows that the mutant yoked hCG binds similarly to the receptor as rhCG, yhCG and HF-hCG. This data reinforces the specificity and usefulness of this antagonist: Mutant yoked hCG binds to the LH/CG receptor but does not activate (acts as an antagonist).

### Example 3

### Binding of hCG to Endometrial and Granulosa Cells

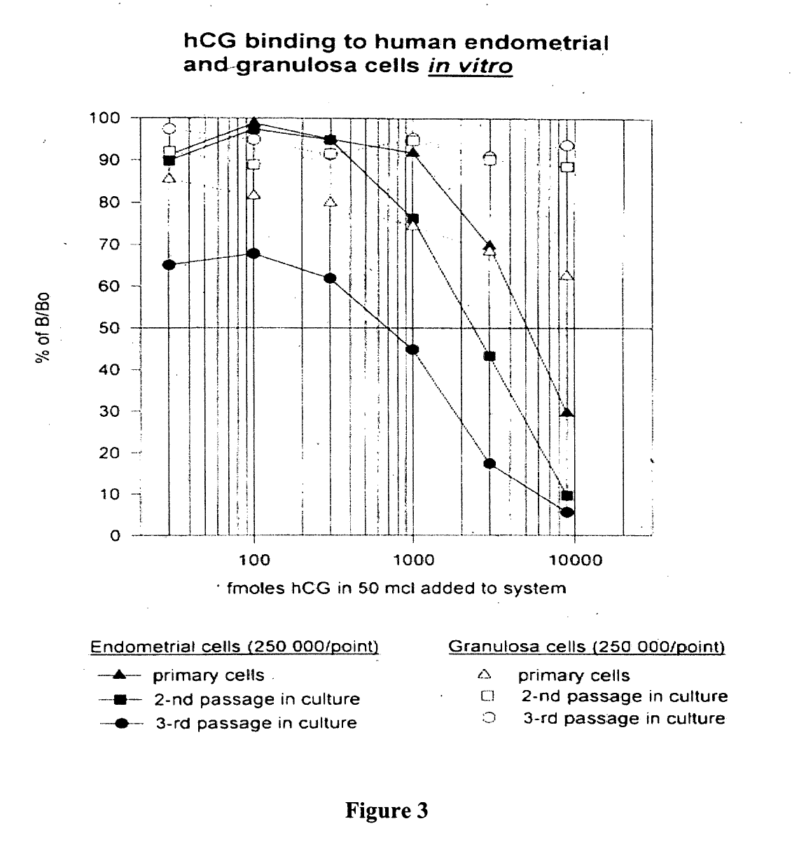

To determine whether human endometrial cells preserve the expression of functionally active LH/CG receptor in vitro, the cell culture suspension was prepared from after mid-phase (4-5 days) human endometrial tissue obtained following curettage. The tissue was ground and incubated in the presence of trypsin (0.25%) and collagenase type I (1.5 mg/ml) for 30 minutes at 37° C. The cell culture obtained by this method represented a mixture of epithelial and decidual cells. Cells were seeded into 6-well plates at 50×10³ cells per cm². To test hCG binding to the cells, they were detached by versene (0.02% EDTA solution in 0.85% saline) for 10 min, washed in serum free Hanks salt solution and incubated in the presence of 100×10³ cpm ¹²⁵I-hCG per 250×10³ cells for 4 h at room temperature. Then cells were pelleted and counted in a gamma-counter. The binding capacity at these conditions was approximately 10-15% of added ¹²⁵I-hCG. To determine the specific nature of hCG binding different amounts of “cold” hCG were added to cells prior to incubation with ¹²⁵I-hCG. After 1 h preincubation at 37° C., ¹²⁵I-hCG was added to cell suspension as described above. As shown in FIG. 3, the highest binding capacity is expressed by primary endometrial cell culture. After two passages the binding decreases, which might be the result either of losing the receptor from the surface or the overgrowth by receptor non-expressing cells, since this is a mixed cell culture. Now endometrial epithelial cells are being separated from decidual cells to determine which of these two endometrial cell subsets is more suitable for testing receptor binding analogs and antibodies. hCG binding to cultured granulosa cells was examined but failed to show significant binding even in the primary cell culture (See FIG. 3). Yet anti-ECD LH-R antisera did recognize freshly harvested human granulosa cells by western blotting. An antibody is tested for binding to cultured granulosa cells. If the antibody detects the presence of receptors in the cultured cells, the non-binding of these receptors to hCG is explored. Alternatively, if the antibody does not detect the receptor, then the receptors may be lost in the process of culturing the cells.

### Example 4

### In-Vitro Bioactivity

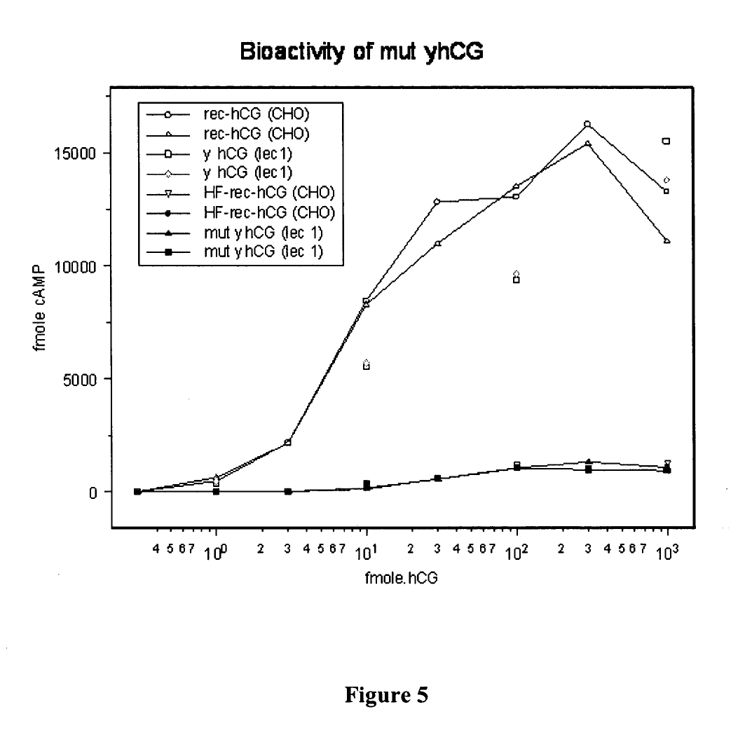

In vitro bioactivity of an hCG antagonist can be measured by the following assay. rhFSH-N2 is an abbreviation for a recombinant human follicle stimulating hormone genetically manipulated to contain an 2 additional asparagine-linked carbohydrate sites. (Weenen et al., Development and comparison of novel long-acting FSH analogues. Journal of Clinical Endocrinology and Metabolism, 2004:89(10):5204-5212.) The rhFSH-N2 was quantified using an hFSH-β specific antibody (Biodesign International, Saco, Me., USA) radioimmunoassay. Biological activity was assessed using CHO cells transfected with the LH/CG receptor (CHO LHr). A total of 2×10⁴ cells were mixed with rhCG, HF-fused-rhCG, yoked hCG or mut-yhCG at varying concentrations in a total volume of 200 μl. Mixtures were incubated at 37° C. for 15 min, and cAMP levels were measured (see results in FIG. 5) using a radioimmunoassay kit (Perkin Elmer Life Sciences, Boston, Mass., USA). The bioactivity of mut yhCG remained low (as expressed in amount of cAMP) while the recombinant hCG bioactivity was high.

### Example 5

### Deglycosylated hCG

A deglycosylated hCG is useful in the methods of the invention. Such a deglycosylated hCG can be identified by having a reduced molecular weight, or as having a different pI value than the wild-type fully glycosylated hCG. For example, deglycosylated hCG can be generated by either site specific DNA manipulation, glycosidase digestion, hydrogen fluoride treatment or expression in specific cell systems which alter post-translational modifications. As an example, bacterial cell culture expression adds no carbohydrate to proteins, while on the other hand yeast and insect expression systems super-glycosylate proteins, and alternatively, mutated Chinese hamster ovary cells (CHO-lec 1 cells, in particular) reduce the amount of carbohydrate added. The amount and type of carbohydrate has a critical effect on the pI of a protein. Therefore a change in carbohydrate type or amount alters the pI profile and mass of a protein. For example increasing the amount of sialic acid as the final carbohydrate moiety on a branch results in a more acidic profile for the protein. pI profile can also be an indicator of a physiologic disorder (e.g., choriocarcinomas produce a more acidic hCG profile).

### Example 6

### Role of a hCG Antagonist in the Treatment of Tumors

This invention provides an example investigating the biological role that hCG, its free subunits, and receptor might play other than induction of steroidogenesis in pregnancy. Preliminary data has been obtained which indicate that hCG and its subunits proliferate cell growth on a number of different cancer cell lines. This observation taken together with the fact that hCG presents in a number of different pathologies other than pregnancy suggests an alternative role for hCG with a possible paracrine/autocrine factor. As hCG or its subunits are secreted by a number of cancers, inhibiting the LH/CG receptor may abolish proliferative activity. This putative growth factor activity of the intact hormone and its subunits will be explored in in vitro models. Anti-receptor antibodies can be evaluated for their ability to inhibit cell growth in these proliferating cells. These antibodies may be useful as diagnostic markers or immunotherapy for certain cancers.

The primary role of hCG is the promotion of steroidogenesis (45-47) yet, the remarkable structural similarity of hCG to a number of growth factors, has prompted an investigation of other functions of the hormone. Indeed, the presence of hCG in non-pregnant physiological conditions suggests additional functions. HCG is synthesized as individual subunits and it is assembled into a heterodimer prior to secretion. A physiological role has only been elucidated for the heterodimer, while the biological significance of free subunits, as well as β-core hCG, has yet to be determined. There is a large body of data that demonstrates the presence of the free subunits and β-core hCG in disease states such as ectopic pregnancy (48), trophoblastic disease (49), and a number of malignant neoplasms (50). As a result, the biological role that hCG, its free subunits, and receptor might perform other than induction of steroidogenesis in pregnancy is being investigated. The seemingly unrelated observations that hCG is structurally related to many growth factors, that the subunits may dimerize in vivo and be active and that many cancers secrete large amounts of hCG or its subunits prompted the exploration of using our anti-ECD LH-R antibodies as possible inhibitors of hCG's autocrine/paracrine activity and therefore potential immunotherapeutic or diagnostic tools as provided for by this invention.

A series of monoclonal antibodies can be isolated which specifically target progressively smaller areas of the binding domain, which can work as more powerful reagents than those currently available.

To summarize evidence suggesting an alternate role for hCG in vivo includes: (i) the abundant presence of free subunits in normal pregnancies and patients with severe neoplastic diseases (50), (ii) mitogenic activity by free subunits of hCG, (iii) the presence of hCG receptors on non-gonadal tissues such as the endometrium, myometrium and fallopian tubes (51-53), (iv) the production of hCG by the embryo at the early blastocyst stage during the preimplantation period (54; 55); and (v) crystallography studies demonstrating a compelling structural relationship between hCG and many growth factors. Also, some neoplasms and cell lines secrete α-subunit and β-subunit (56) as putative autocrine factors and, in this regard, it has been shown that the addition of anti-α-subunit antibodies can inhibit the growth of lung carcinoma cells in vivo and in vitro (57). Existing data appears to support a likely autocrine/paracrine role for hCG and its subunits.

The experiments were designed to show that hCG and its subunits are growth factors or inducers of cell differentiation. HCG can promote differentiation of cytotrophoblasts into syncytiotrophoblasts (58), and free α-subunit stimulates prolactin production by the decidua (59). The production of hCG by the early blastocyst (60-64) suggests that hCG promotes the viability of the embryo and facilitates implantation. The presence of receptors for hCG on the endometrial surface (52; 65) suggests that it supports implantation by reprogramming endometrial cells, blocking apotosis (66), which normally follows the regular menstrual cycle, so the secretory phase may continue (67). The potential role of hCG on implantation is explored by testing its effect on endometrial cells in vitro.

It is thought that neither the α- or β-subunit individually bind the LH/CG receptor, and that both subunits are required for activity (34). Nonetheless, a β-homodimer of hCG binds the receptor with a relatively high affinity and is biologically active (68). The α-homodimer induces cAMP activity. Others have demonstrated that unique combinations of the various loops of the α- and β-subunits have an affinity for the hLH/CG receptor and can induce cAMP production (69). Therefore, there is a body of evidence that suggests the individual subunits themselves may play a role in activating the hLH/CG receptor in either a physiologic state or a disease state where ectopic production of a large amount of a single subunit has been identified. Polyclonal anti-receptor antibodies have been produced to facilitate purification of our expressed receptors and a panel of monoclonal antibodies will be produced. These antibodies are important reagents for determining if proliferation can be blocked by their addition. This potential attribute of an anti-receptor antibody becomes especially important in light of the role of hCG in neoplastic growth. This putative function will be studied by testing our anti-receptor antibodies or non-glycoylated hCG for competitive binding to a variety of cell lines that express and/or require hCG subunits for growth. In addition, retrospectively screening pathology carcinoma samples to determine if there is a possible diagnostic use for the antibodies of this invention is being undertaken.

It is believed that an understanding of the three-dimensional structures of both the LH/CG receptor and the activating hormone (hCG) are necessary to the design of simple pharmacologic agents that will exploit such interactions. Our group in earlier studies contributed substantially to the field by producing the crystals of hCG and solving the structure by x-ray diffraction studies. Hormone analogues will be designed and constructed to control these interactions. Furthermore, uncovering additional functions of the hormone that may allow pharmacological intervention in the course of this proposal.

Anti-receptor antibodies produced by the inventor will be utilized for the screening of neoplastic disease or as a potential inhibitor of cell growth. Biochemical analyses will be used to identify smaller ECD/hCG complexes that will be used as substrates for crystallography studies and possibly as antigens to generate a different panel of anti-receptor antibodies. The invention provides reagents such as antibodies that could have significant clinical applications.

Reproductive biologists believe that hCG is biologically active as the dimer. Interestingly there appears to be an abundance of free subunits during many non-pregnant pathologies. This phenomenon has puzzled researchers. As described above, the inventor and others have identified a number of cell lines which respond proliferatively to α or βhCG presence in the medium. Data independently derived from the crystal structure indicating a remarkable structural similarity to the growth factors presented the first physical relationship for an alternate function for hCG or the subunits as possible autocrine/paracrine factors. The invention provides for the following related to hormone and the subunits: (i) As hCG is thought only to be active as the dimer and adding free subunit to certain cells stimulates growth, is there a discrete receptor for each subunit? (ii) The invention provides for the finding that the β-homodimer and the α-homodimer both are capable of inducing cAMP activity leading to the question of whether the subunits dimerize in vivo and bind to the hCG receptor. (iii) The invention provides for subunits that have a proliferative effect on certain cell lines; it will be determined if hCG has a similar stimulatory activity. (iv). If hCG stimulates cells, does adding an anti-LH/CG receptor antibody inhibit growth? (v). Do tumors which secret hCG have the receptor on their surface? (vi). Could immunohistochemical screening of pathology cases with anti-receptor antibody be a diagnostic tool or a predictor of outcome?

Proliferative Activity:

To study growth and differentiation activities of hCG and its subunits, cell lines described by Chard and Iles (56) are used. These cell lines are known for their ability to produce free β-hCG and their growth dependence on this subunit. Since it has been shown that urothelial neoplastic cell lines, such as the transitional bladder carcinoma cell line SCaBER, secrete high levels of free β-hCG (56) this cell line is used as an in vitro model system for testing the effect of β-hCG (and β-homodimer) on growth activity. In addition, cell lines that produce low or undetectable amounts of β-hCG in vitro are used, although the tumor of origin of these cells produces high levels of β-hCG in vivo. The invention provides for β-hCG as both an autocrine and paracrine factor. Division of the cell lines into two groups occurs: 1) cells which produce free subunits both in vivo and in vitro and 2) cells which do not produce any detectable amount of free subunits in vitro, but their growth apparently depends on the presence of hCG subunits in vivo. Group 1 includes urothelial neoplastic cell lines SCaBER and TccDeS as described by Chard and Iles, and ‘normal’ cells of transitional bladder cell epithelia HU609 (56). Group 1 also includes neoplastic bladder cells 5637 (intermediate producer) and T24 (low producer). Group 2 consists of the germ tumor cells TERA I and TERA II, both known to originate from tumors where high levels of β-hCG are detected in vivo but do not secrete detectable levels of β-hCG in vitro. (114). For the effect of free α-hCG (and α-homodimer) on growth activity, the human lung tumor cells, ChaGo-K-1, which originate from bronchogenic squamous cell carcinoma, described by Talwar (57) as the model for the effect of α-hCG on growth activity, are used. It was shown that the growth of ChaGo-K-1 cells strongly depends on endogenous α-subunit, and that in the presence of anti-α-antibodies cell growth is significantly diminished. This cell line is used as a positive control for α-hCG producer/responder cells in the studies on α-hCG growth activity.

Methods: Several methods are utilized to identify the role of the free subunits on cell growth. One is the use of specific polyclonal and monoclonal antibodies, that bind and neutralize hCG subunits in cell culture. As Talwar (57) has demonstrated with ChaGo-K-1 cells, the addition of anti-subunit polyclonal and/or monoclonal antibodies to cells that produce or respond to either subunit will most likely inhibit cell growth by preventing subunits from binding to the cells. To rule out the cytotoxic effect of the antibodies or of the immune complexes, non-hCG polyclonal and monoclonal antibodies are used as controls, such as anti-fibrinogen, or anti-IgG antibodies. Another approach used to identify the growth capability of free subunits is to employ analogs of hCG subunits which have been altered at specific residues. This may identify residues within the sequence essential for growth activity. A large number of mutants were described elsewhere (115; 116). These mutants are added to the cell lines, which respond to unaltered subunits. Finally, the anti-ECD antibodies are used to determine whether they can compete with the subunits or if they can bind simultaneously. If the antibodies can bind simultaneously, or if they do not bind at all, it may indicate the presence of distinct subunit receptors on cells responding to α or β subunit.

Effect of free subunits on normal tissues. Since it has been shown that some primary cultures can secrete free subunits (HU609 secretes β-hCG) it is possible that free subunits may direct cell proliferation in different tissues. The growth stimulation effect of free subunits on human endometrial cells, human vascular endothelium cells and ovary granulosa cells is tested. A primary cell population is isolated from these tissues and culture them in vitro. The stimulation of replication and cell growth is evaluated using propidium iodide staining and flow cytometry where the percentage of cells entering the DNA synthesis phase (replicative phase of cell cycle, S-phase) is measured. A colorimetric assay is also used with the tetrazolium salt MTT (117; 118), as a more practical alternative to measure proliferation. From this study, estimation of the role of free subunits as paracrine growth factors is determined.

Growth response to free subunits. Continued growth of cells in culture balances two opposing processes; cell replication and cell death. The growth promotion activity of free subunits involves the stimulation of cell proliferation and/or an inhibition of cell death (apoptosis). Two different methods are employed. Using cell lines or tissues previously determined to respond to either subunit, the accumulation of BrdU (BromodeoxyUridine) in a cell population is first measured by using FITC (fluorescein isothiocyanate) labeled antibodies to BrdU and flow cytometry (119). The rate of accumulation of BrdU in cell populations treated and non-treated with free subunits shows if the fraction of dividing cells increases in the presence of free subunits. If the fraction of dividing cells is the same, while the cell number increases, this suggests the role of free subunits as an anti-apoptotic factor. However, it is also possible, that both effects, proliferative and anti-apoptotic, occur, since it was shown that many classical growth factors can also protect cells from apoptosis (120). Therefore, the activation of anti-apoptotic genes, such as bcl-2 (121; 122), is tested by examining their expression in target cells.

Elucidation of the Growth Activity Mechanism for hCG and its Subunits. Identification and Characterization of Specific Receptor(s).

Since it has been shown that certain bladder carcinoma cells (114) respond to β-hCG and it has also been demonstrated that certain lung carcinoma cells (57) respond to α-hCG, there should be a mechanism through which the effects are mediated. It is unclear if there are specific receptors for free β-hCG and free α-hCG which mediate signal transduction. It has been shown that the homodimers of the α- and β-subunits bind to the LH/CG receptor and stimulate cAMP activity. It is possible that, for cells responding to α or β, the signal is mediated through the LH/CG receptor. This is tested hypothesis by determining if the anti-ECD antibodies can block proliferation (see below). Alternatively, if there are specific subunit receptors, these receptors are identified in two ways: First, the specific β-hCG and α-hCG receptor(s) are identified by labeling the ligand with ¹²⁵I and measuring binding to the responding cells or their membranes. Since both subunits have large molecular masses inhibiting diffusion through the cell membrane (β-hCG-22 kD and α-hCG-14 kD), if the putative receptors exist they are probably extracellular. Therefore, the binding can be measured to the whole cells or to an enriched fraction of cellular membranes. Since free subunits as growth factors are considered, it is unlikely that the number of specific receptors on the cell surface will be high (123). As a result, to detect binding a large number of cells is used for a single measurement (10⁶-10⁷). Therefore, binding experiments using membrane fractions might be more effective. Competition experiments with ‘cold’ subunits provide evidence of the specificity of binding and estimate the affinity of binding (Scatchard plot) and number of receptors on the cell surface.

Furthermore, the invention provides for the characterizing and isolating of the putative subunit receptors, starting with the β-subunit receptor. In order to do so, membrane fractions are prepared and coprecipitated using labeled ¹²⁵I-β-hCG and anti-β-hCG antibodies. Following treatment with glutaraldehyde, to cross link the receptor and ligand, the material is run on SDS-PAGE. Although there will likely be a substantial amount of crosslinking between receptor, ligand and antibody, the autoradiography should also reveal a band corresponding to the receptor/ligand complex from which one can deduce the molecular weight of the receptor by subtracting the known molecular weight of β-hCG. Responding cells are also grown in the presence of ³⁵S-methionine and ³⁵S-cysteine, β-hCG is added, and the complex is precipitated with the anti-β-hCG antibodies. SDS-PAGE and autoradiography on this material enable one to locate the receptor band itself. Another approach to locate the receptor is to isolate membrane fractions from the responding cells, extract the membrane proteins with Triton X-100 and to run the PAGE in non-reducing conditions (124). Gels are transferred onto nitrocellulose membranes and incubated with ¹²⁵I-labeled ligand. This ligand blotting technique (125), can also reveal the location of the actual receptor and its molecular weight. The specificity of ligand binding is tested by adding increasing amounts of non-labeled subunits. To isolate the receptor, a ligand-sorbent column will be made, by immobilizing free subunits on CNBr-Sepharose 4B and passing through it the solubilized membrane fractions from responding cells. The eluted material is compared with the material identified through ligand blotting and amino acid analysis is performed. Once there is a sufficient peptide sequence, oligos are designed for the isolation of a cDNA clone that encodes the receptor by RT-PCR.

The invention provides for antibodies to the putative subunit specific receptor. Animals are immunized either with the material eluted from the ligand-sorbent column, or the corresponding band from the gel is cut and used as an immunogen. Antibody development is monitored by RIA.

Once antibodies that are specific for the subunit receptors are produced, they are utilized to isolate a cDNA of the subunit receptor gene. Labeled antibody is used to screen a cDNA expression library constructed in λgt11 from mRNA isolated from responding cells. Positive clones are plaque purified, the cDNA inserts are sequenced and their translation product is compared against the NIH database of known proteins and hypothetical translation products of cloned sequences. The specificity of binding to the λgt11 expressed protein is also determined by competition with cold subunits. Finally, these cDNAs are subcloned into eukaryotic expression vectors, transformed into NIH 3T3 cells, and the cell lines are tested for their ability to bind free subunit.

The Use of Anti-ECD Antibodies to Identify the Presence of LH/CG Receptors in Various Tissues, and to Block Proliferation of Cells Responding to hCG or its Subunits.

Diagnostic Indicator: In general, germ cell tumors, sex cord-stromal tumors, and gestational trophoblastic tumors have hLH/CG receptors and/or the potential of secreting hCG. Immunohistochemical visualization of the hLH/CG receptor with our polyclonal anti-ECD antibodies, on theca and granulosa cells in normal human ovaries, and leydig cells in normal testis, has been described. The invention provides for histological sections from archived paraffin blocks of benign and malignant tumors of the female and male genital tract to be examined to determine whether the anti-ECD receptor is present. The invention further provides that case series of selected tumors are collected and evaluated to determine whether receptor status has a significant prognostic impact. The following is a preliminary list of histology slides obtained by the inventor: (i) granulosa cell tumor of ovary, (ii) theca cell tumor of ovary, (iii) sertoli cell tumor of ovary, (iv) sertoli-leydig cell tumor of ovary, (v) leydig cell tumor of ovary, (vi) dysgerminoma of ovary, (vii) choriocarchinoma of ovary, (viii) hydatidiform mole, (ix) choriocarcinoma of uterus, (x) seminoma of testis, (xi) choriocarcinoma of testis, (xii) leydig cell tumor of testis, and (xiii) sertoli cell tumor of testis.

Immunotherapy: The invention provides for the examination of established human cancer cell lines, such as JAR (human choriocarcinoma) (126) and OCC1 (human ovarian cancer) (13) to determine if adding anti-ECD antibodies inhibits proliferation or induces apotosis. The invention further provides that these studies serve as a precursor to future studies utilizing xenograph tumor models in mice.

Defining an In Vitro Role for hCG or its Subunits in Facilitating Implantation on Endometrial Functions.

The invention provides for the elucidation of a direct role of hCG and its subunits on endometrium epithelial cells (in addition to its role in maintaining ovarian steroid synthesis), the primary maternal cells making contact with the pre-embryo (127; 128). Purified epithelial cells are cultured from human endometrium. From in vitro studies on hCG production by pre-implanted blastocysts, the blastocyst begins producing hCG at 160-170 h post fertilization (129; 130). At this time blastocyst activation is completed and implantation starts. During the initial phase of implantation, the trophoblasts attached to the receptive epithelial cells, and the uterus closes around the blastocyst (131). Epithelial cells signal the blastocyst to recruit vast numbers of different factors, such as cell adhesion proteins and lectins, cytokines and growth factors (132-135). The role of maternal factors, such as progesterone and estrogen in priming the uterus for implantation is well established (136), while the role of factors expressed and secreted by pre-embryos and their contribution to the implantation is poorly understood. Since hCG (and apparently free β-hCG (54)) is one of the earliest pre-embryo products, the invention provides for revealing the role of these molecules in the initial steps of implantation where endometrial cells are the primary target. The invention provides for considering several potential effects of hCG and free β-subunit on endometrial epithelium: 1) growth stimulation, 2) induction of apoptosis and 3) induction of secretion of several endometrium specific proteins, such as PP-12, PP-14, prolactin and CA-125 (137; 138).

Endometrial cell culture: The invention provides for obtaining samples of human endometrium during non-conceptual menstrual cycles from women undergoing diagnostic curettage for gynecological observations. Cell suspensions are prepared from hysteroscopy or hysterectomy samples obtained from non-malignant uteri and culture them according to previously described methods (139). Briefly, the tissue is trimmed in DMEM, supplemented with 20% fetal calf serum, glutamine and antibiotics. The single cell suspension is separated from clumps by allowing the latter to settle by gravity. The precipitate is treated with collagenase type II (0.05 mg/ml) for 2 hours and the single cell supernatant is collected and combined with the first portion. The supernatant is seeded to tissue culture dishes (100 mm) for 2 h at 37° C., to allow fibroblasts to attach. The unattached cells are transferred to new TC dishes at 2×10⁵ cells per ml. Since it has been shown that there are LH/CG receptors on the endometrial epithelium (the invention also provides for confirming that the anti-ECD antibodies bind these receptors), these cells are tested for their ability to respond with cAMP synthesis following addition of hCG.

Physiological responses of endometrial endothelia to the treatment by hCG and free subunits: The culture media is supplemented by adding hCG and its free subunits and monitors a number of parameters, with known relevance to the implantation process. Using commercially available kits the levels of prolactin are measured (modified method), PP-12, PP-14, and CA-125 (140; 141). The invention provides for the study of the cumulative effect of estrogen and hCG and β-hCG on the endometrium. As a control estrogen is used to monitor the same parameters. The presence of specific receptors for free β-hCG is also monitored using methods described above. The effect of hCG and β-hCG on growth and apoptosis of these epithelial cells is also monitored.

### Example 7

### Mutated and Yoked hCG

### An hCG Antagonist

Women that are undergoing an IVF cycle are potentially candidates for ovarian hyperstimulation syndrome (OHSS) will not necessarily have to have their cycles cancelled. Most commonly during that protocol women are given FSH for 8-12 days followed by hCG in the luteal phase. OHSS is the most serious complication of IVF. Despite careful monitoring, a small number of women (about 3-5% of the treatment cycles) may develop OHSS. It is a small percentage but when the total number of women undergoing IVF is considered, it is a rather large number nonetheless. Any patient undergoing ovulation induction is at risk of developing OHSS, although some more than others. Ovarian hyperstimulation syndrome may be classified as mild, moderate or severe by symptoms and signs. The worst cases tend to be associated with pregnancy. Severe OHSS is a life threatening complication following ovarian stimulation.

The symptoms usually begin 4-5 days after the egg collection. The majority of women have a mild or moderate form of the syndrome and invariably resolve within a few days unless pregnancy occurs, that may delay recovery. Patient may complain of pain, a bloated feeling and mild abdominal swelling. In a small proportion of women, the degree of discomfort can be quite pronounced. In some cases cysts appear in the ovaries (ovarian cysts) and fluid may collect in the abdominal cavity causing discomfort.

Very rarely, in about 1-2% of cases the ovarian hyperstimulation is severe and the ovaries are very swollen. The woman will feel ill, with nausea and vomiting, abdominal pain. Fluid accumulates in the abdominal cavity and chest, causing abdominal swelling and shortness of breath. Reduction in the amount of urine produced. These complications require urgent hospital admission to restore the fluid and electrolyte balance, monitor progress, control pain and in some very serious cases, termination of pregnancy. Complications associated with severe OHSS include blood clotting disorders, kidney damage, twisted ovary and in some cases, death.

Causes of OHSS include over-response to fertility drugs. The cause is unknown. Women at risk of developing OHSS include: women with polycystic ovaries; younger women; high estrogen hormone levels and a large number of follicles or eggs; administration of GnRh agonist; and the use of hCG for luteal phase support.

Management and treatment options for OHSS currently include withholding hCG administration when the blood estrogen levels and ultrasound scans show a high risk of severe OHSS; proceeding with egg collection and insemination, but have any embryos frozen and not proceeding to fresh embryo transfer in that cycle and undergoing subsequent a frozen embryo transfer treatment cycle; or coasting to stop with the gonadotropin stimulation and continuing the agonist suppression until estrogen levels decline to acceptable levels before proceeding to egg collection.

For the potential mild to moderate OHSS patient (not severe) one gives hCG to trigger ovulation and then gives the hCG-antagonist to stop the progression of the syndrome.

An alternative application for the product is when the hCG-antagonist is given to induce an abortion or as a contraceptive agent. Although unlike the steroid analogues that are in oral contraceptives this analogue would have to be injected or implanted like, for example, Norplant.

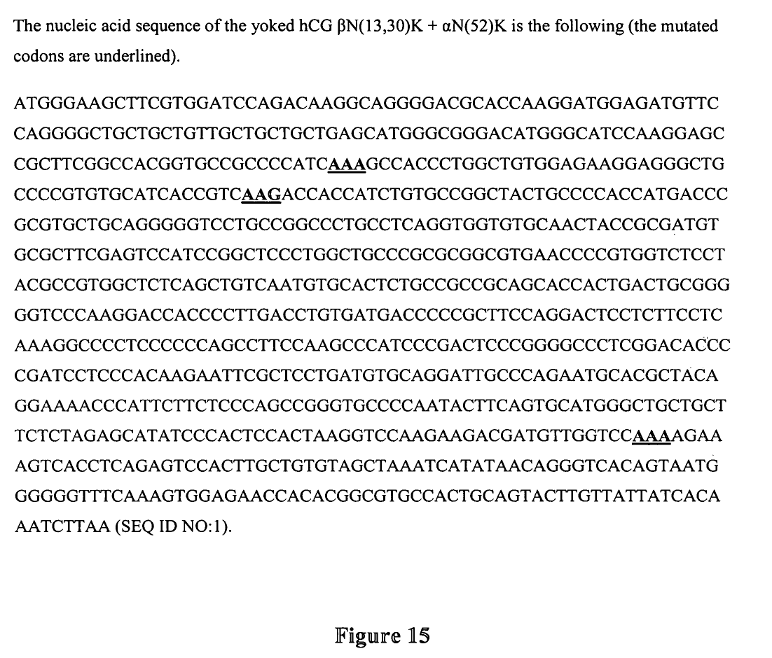

Rec-hCG (CHO) (recombinant hCG that is fully glycosylated and biologically active, expressed as a dimer in Chinese hamster ovary cells (CHO)); y hCG (lec1) (the alpha and beta subunits are ligated (yoked) expressed in lec-1 cells (cell that was genetically altered so that expressed proteins are not fully glycosylated; HF-rec-hCG (CHO) the native protein is chemically deglycosylated by a very harsh treatment (hydrogen fluoride) removes 70% of the N-linked carbohydrate; mut y hCG (lec1) the yoked hCG has had 3 of the 4 N-linked carbohydrate sites altered by site specific mutagenesis (FIG. 15) and then is expressed in lec1 cells.

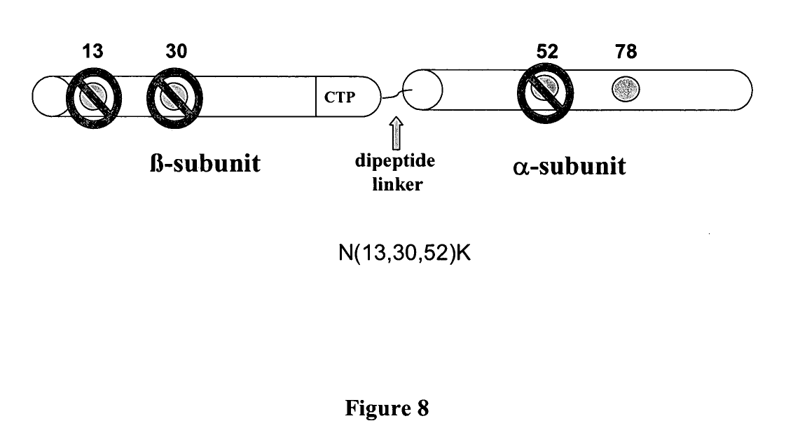

It was thought that expressing rec-hCG in lec1 cells would be enough to ablate hCG activity. The invention is directed to an hCG antagonist, i.e. an hCG molecule that would bind to the LH/hCG receptor but would not induce activity. For hCG, the Asn 52 carbohydrate site on the alpha subunit is critical for activity. HF-rec-hCG (CHO) has been shown to be an hCG antagonist but HF-hCG cannot be injected into humans because the hydrogen fluoride (which is deadly) cannot be completely removed. On the graph in FIG. 5 there is no cAMP produced which is the endpoint for this activity profile. The graph in FIG. 5 shows there is a substantial rise in cAMP expression not as great as native (rec-hCG (CHO)) but still quite significant. Three of four the N-linked carbohydrate sites in hCG were specifically removed to generate mut y hCG (lec1) to get the hCG antagonist (FIG. 8).

This example provides (a) a recombinant hCG that is fully glycoslyated and biologically active, expressed as a dimer in CHO cells, and (b) yoked hCG (wherein the alpha and beta subunits are ligated or yoked). N-linked glycosylation of hCG ligand is a requirement for receptor activation, not binding. There are four N-linked glycosylation sites in hCG. Three of these sites, at βN13, βN30 and αN52, influence receptor activation (Matzuk et al., JBC 254(5):2409-2414 (1989)). A mutant of yhCG that is resistant to N-linked glycosylation at βN 13, βN30 and αN52 was prepared in the following manner. An antagonist mutated and yoked hCG has three of the four N-linked carbohydrate sites altered by site-specific mutations. One site that is altered is Asn 52 since it is critical for activity. In this embodiment, two other sites that are altered are on the beta-subunit, and they are 13 and 30. The alpha site 52 is the site most identified with bioactivity. Asn 78 on alpha is the only N-linked site that was kept intact in this embodiment. This site appears to be critical for binding to the receptor but not important for activity.

The coding sequence of yoked hCG-1, contained within the baculovirus expression vector pVL1393 was used. The insert encodes the leader peptide and mature β-chain sequence of human chorionic gonadotropin followed by the coding region of the mature α-chain. The two coding regions are linked to each other, in frame, by the hexanucleotide, GAATTC. The yhCG insert was excised from pVL1393 and inserted into the subcloning vector, pLit28 (New England Biolabs, Waverly, Mass.) to facilitate its subsequent manipulation. Mutations were introduced into the aforementioned positions using the Quik-Change Site-Directed mutagenesis kit (Stratagene, Torrey Pines, Calif.). Each asparagine codon was converted to a lysine codon by single nucleotide substitution. The βN(13)K and βN(13, 30)K mutants were prepared sequentially, the latter derived from the former. The αN(52)K mutation was prepared separately. The triple mutant, βN(13, 30)K+αN(52)K, was prepared by ligating together restriction fragments containing the βN(13, 30)K and αN(52)K mutations into pLit28. The integrity of the yhCG βN(13, 30)K+αN(52)K triple mutant coding sequence was confirmed by di-deoxy chain termination sequencing. The mutant was sub-cloned into the mammalian expression vector, QCXIN (BD Biosciences Clontech, Palo Alto, Calif.) and introduced into Lec 1 or CHO-K1 cells by Lipofectamine-mediated transfection (Invitrogen, Carlsbad, Calif.). Individual colonies were obtained by selection in the presence of one mg per mL G418 sulphate and recovered from the dish using cloning cylinders. Detection of gene expression was accomplished by examining cell culture supernatants for the presence of yhCG in western blot using anti-hCG β chain antibody as probe.

The nucleic acid sequence of the yoked hCG βN(13,30K+αN(52)K is shown as SEQ ID NO:1 in FIG. 15.

### Example 8

### Effect of a Novel hCG Antagonist on Suppressing Ovulation in Rats

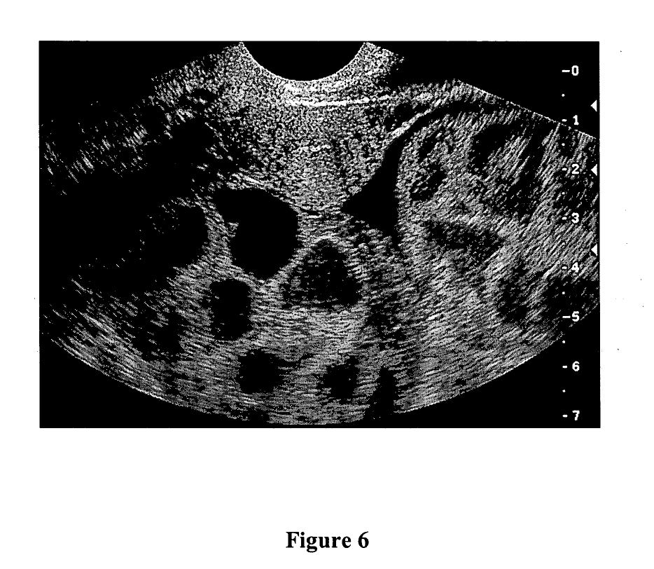

Stimulation and induction of multifollicular growth by gonadotropins remains the first step in any standard stimulation protocol used for assisted reproductive technology (ART), for example in vitro fertilization (IVF). However, it is well known that use of these drugs is not without side effects. Ovarian hyperstimulation syndrome (OHSS) is a condition that can occur in women undergoing in vitro fertilization, after treatment with gonadotropins and human chorionic gonadotropin (hCG) to stimulate oocyte growth and maturation. Clinical manifestations of OHSS include enlarged ovaries, the production of vasoactive substances, and an increase in vascular permeability, leading to a significant loss of fluid into the extravascular space. This causes pleural and pericardial effusions, ascites, and generalized edema (FIG. 6). In severe cases, OHSS can lead to prolonged hospitalization and multi-organ failure. Currently, treatment is limited to supportive care. Current treatment modalities for OHSS focus on supportive care, including intravenous fluids, thromboprophylaxis, and the judicious use of colloids (i.e., albumin) and diuretics to maintain peripheral perfusion. Occasionally, in severe cases, abdominal paracentesis is required.

Incidences of OHSS have increased worldwide due to the expansion of assisted reproduction techniques. Although significant OHSS has a relatively low incidence of 2%, it may in severe cases (0.1-0.2% of IVF cycles), result in a potentially life-threatening situation, requiring hospitalization with intensive care monitoring due to multi-organ dysfunction. It is characterized by a cascade of events that leads to increased capillary permeability with the loss of fluid into the third space, resulting in hemoconcentration, electrolyte imbalance, and a predisposition for thrombosis.

Although the etiology of OHSS is still unclear, it is thought that hCG, whether exogenous (to induce final oocyte maturation during IVF) or endogenous (pregnancy-derived), is the triggering factor. In fact, when hCG is withheld or replaced by an endogenous surge of LH induced with GnRH analogues, the incidence of OHSS is dramatically reduced or eliminated. Two distinct patterns of OHSS exist. The early type occurs 3-7 days after ovulation triggering by hCG and the late type, that occurs 12-17 days after hCG. Early OHSS is an acute consequence of the exogenous hCG administration before oocyte retrieval and is usually related to an excessive ovarian response to gonadotropin stimulation. Late OHSS is induced by endogenous hCG from the initiated pregnancy and is observed only in patients who become pregnant, especially in those with multifetal gestations.

Some women have a greater risk of developing OHSS. These patients are usually young (<35), have a low BMI, are oligomenorrheic and anovulatory and respond to exogenous gonadotropins with estradiol (E2) levels greater than 3000 pg/mL and multiple follicles (>20). Egg donors and patients with polycystic ovarian syndrome are at highest risk for OHSS. Other OHSS risk factors include an hCG trigger, a history of OHSS, or inherited or acquired thrombophilia.

The mechanism by which hCG increases vascular permeability is not fully understood, but it is known that the vascular endothelial growth factor (VEGF) system plays a pivotal role in the pathophysiology of OHSS. Several isoforms of VEGF have been found in many studies to be elevated in both early and late OHSS. The period following hCG administration also reveals clinically significant alterations in the coagulation and fibrinolytic systems, including elevations in Factor 2, 5, 7, 8, and 9 levels which occur within 2 days of treatment continue for over 3 weeks and longer, if pregnancy is established. These results suggest that hCG plays a key role in the pathogenesis of OHSS.

Development of an hCG Antagonist to Reduce OHSS

The invention provides an antagonist to hCG activity by using molecular biology to produce an hCG that binds strongly to the LH receptor without activating the receptor. In blocking hCG activity, ovulation will be significantly diminished making this an effective adjuvant therapy for treatment of OHSS.

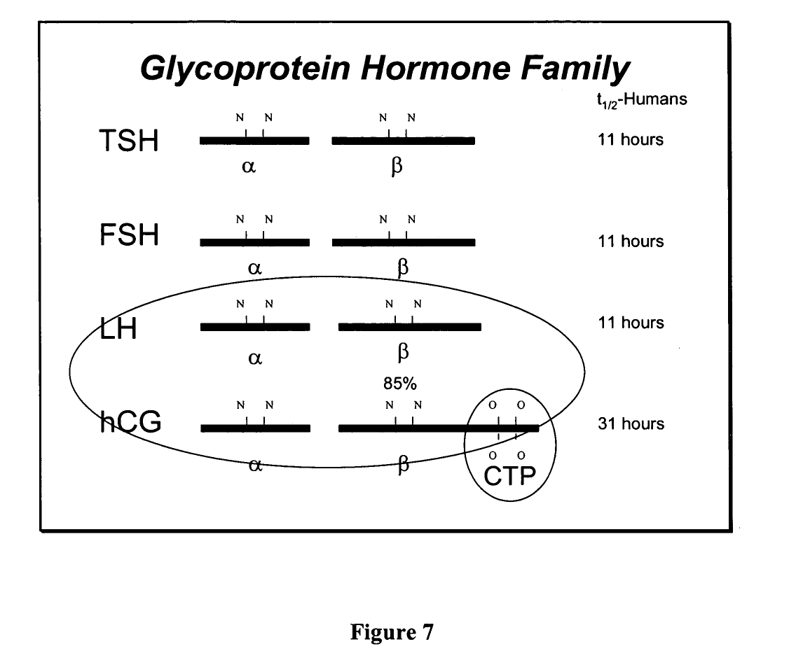

The O-linked oligosaccharides account for the long elimination half-life of hCG in humans (31 hours) compared to the other glycoprotein family members (11 hours) by reducing glomerular filtration within the kidney (FIG. 7). O-linked oligosaccharides regulate the half-life (t_{1/2}) of hCG. Chemical removal of the O-linked carbohydrates or genetic removal of the CTP sequence which signals O-linked carbohydrate addition revealed that hCG was still able to bind to its receptor and stimulate biological activity, however, the half-life was significantly reduced (FIG. 7). In contrast, N-linked oligosaccharides are essential for hCG hormone function. Loss of the beta chain N-linked carbohydrates at Asn 13 and 30 reduces beta subunit secretion and dimerization and weakly contribute to receptor activation. On the alpha subunit, the oligosaccharide at Asn 78 assists subunit dimerization and enhances the ability of hCG to bind tightly to the receptor. The oligosaccharide at Asn 52 is essential for maintaining protein integrity within the cell and assisting in protein secretion. More importantly, however, Asn 52 is critical for normal LHR activation. hCG can be converted from an agonist to an antagonist by removal of the N-linked oligosaccharides.

Development of an hCG Antagonist by Site Directed Mutagenesis

FIG. 8 is a diagram of a yoked hCG-antagonist provided by the invention. Site-directed mutagenesis was used to develop a yoked-hCG antagonist with removal of three of the four N-linked glycosylation sites while keeping one site intact, Asn 78, on the alpha subunit to enhance receptor binding. The y-hCG-Ant was designed to competitively bind the hCG receptor without activating the receptor, both in vitro and in vivo. The alpha and beta subunits were yoked together as a single contiguous protein. Chinese hamster ovary (CHO) cells were engineered for production of the yoked-hCG (y-hCG). Asn 52, 13 and 30 were removed to prevent LH receptor activation. Asn 78 was not altered. The effect is that the hCG antagonist binds but does not activate the receptor.

Ovulation Protocols

Rats were superovulated with 50 IU of pregnant mare serum gonadotropin (PMSG), the standard murine superovulation agent, for four days, followed by 30 IU of hCG or hCG antagonist, or 0.1 mL of saline on day 5. After 48 hours, rats were sacrificed and the ovaries were removed for gross and histologic examination.

Rats were then treated with a conventional ovulation protocol. They received 10 IU of PMSG for one day, followed by 10 IU of hCG, hCG-Ant, or saline 48 hours later. Sixteen hours after hCG treatment, rats were sacrificed and the ovaries and fallopian tubes were removed and examined under a dissecting microscope to determine the number of ovulated oocytes.

In Vitro Studies

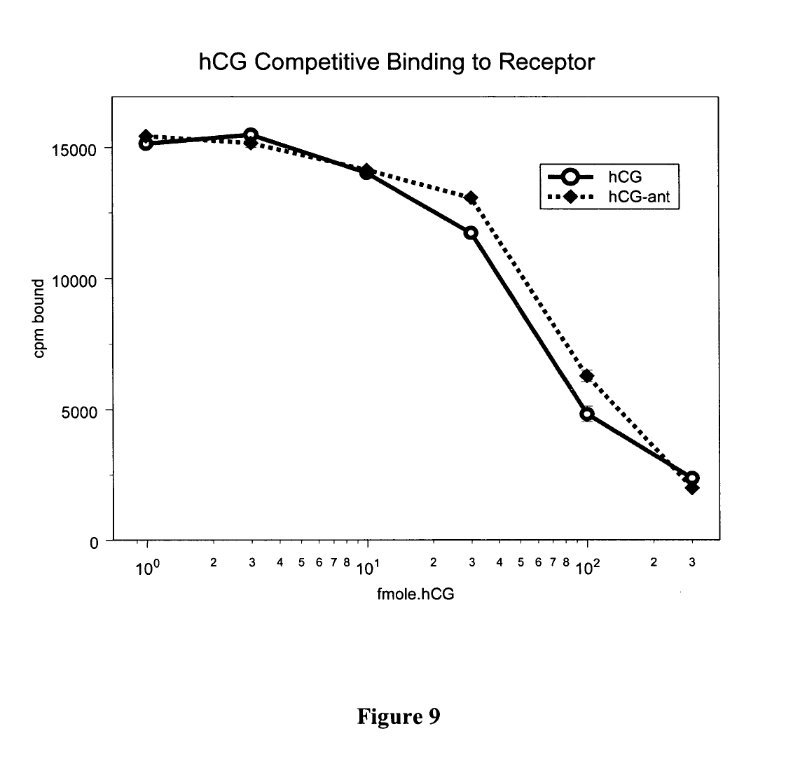

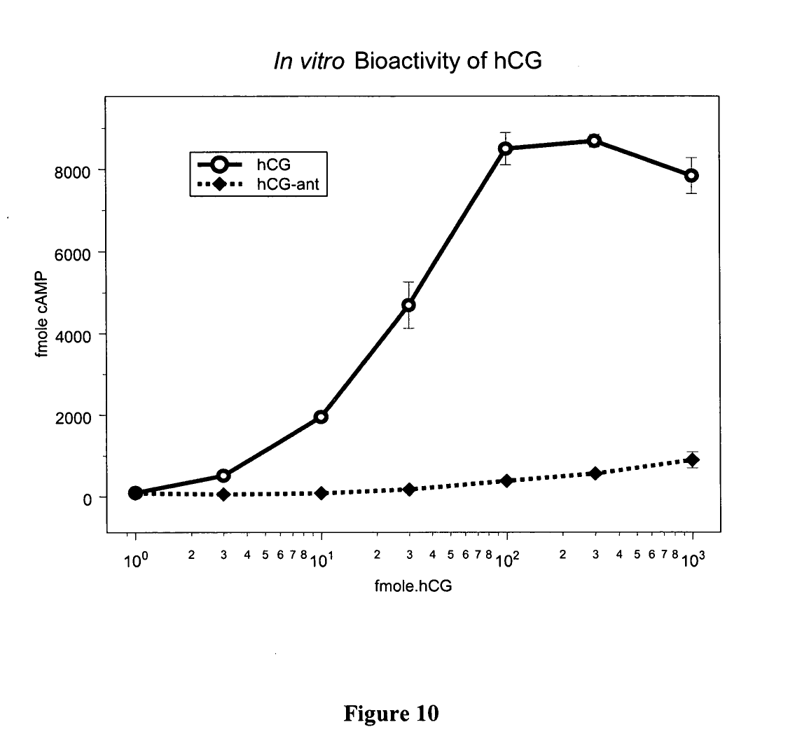

In vitro studies consisted of receptor binding studies and cAMP bioactivity studies. These studies show competitive binding of hCG-Ant to LH receptors in Chinese hamster ovary cells expressing the LH receptor (CHO-LHR) (FIG. 9). HCG receptor activation in vitro was assessed by measuring cAMP activity in CHO-LHR cells (FIG. 10). hCG-Ant did not stimulate cAMP levels above baseline. In vivo studies used immature female Wistar rats (21d) randomized into 6 groups. Ten rats received 50 IU pregnant mare serum gonadotropin (PMSG) ip, the standard for murine superovulation, for 4 days. Four rats received 30 IU hCG (Pregnyl) to induce OHSS (PMSG/hCG), 4 rats received 30 IU of hCG-Ant (PMSG/hCG-Ant) and 2 rats received 0.1 mL of saline (PMSG/Sal) on day 5. The control group (Sal/Sal n=2) received 0.1 mL saline ip for 4 days followed by 0.1 mL saline on day 5.

In Vivo Studies

In vivo studies consisted of an ovulation studies in immature female rats and vascular permeability studies in a rat OHSS model. In vivo effects of hCG and hCG-Ant alone were assessed by injecting 4 rats with 0.1 mL of saline for 4 days followed either by 30 IU of hCG (Sal/hCG) or hCG-Ant (Sal/hCG-Ant) on day 5. Serum estradiol and progesterone levels were measured at 48 hrs after hCG, hCG-Ant, or saline treatment. Rats were then sacrificed. Ovaries, uterus, heart and lungs were prepared for gross examination and histologic evaluation.

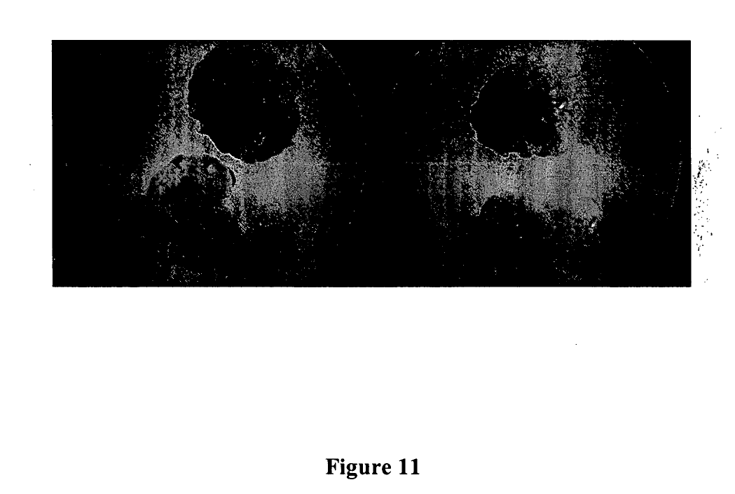

Pharmacodynamics were evaluated by comparing serum estradiol and progesterone, number of ovarian surface stigma (surface remnant of ruptured follicle), weights and histologic appearance of the ovaries between the different treatment groups. Ovarian weight, estradiol and progesterone levels were not statistically different between the PMSG treated groups. The ovaries from the PMSG/hCG superovulated rats contained significantly more ovarian stigma, or remnants of ovulated oocytes than those treated with PMSG/hCG-Ant (FIG. 11). This difference was apparent both on the surface of the ovaries, and by serial sectioning. HCG-Ant reduced the number of surface stigma by 70%, and the number of stigma by serial section by 50% (Table 1). There was an 80% decrease in the number of ovulated oocytes in rats treated with hCG-Ant (Table 1). The number of ovulated oocytes in the fallopian tube was determined under a dissecting microscope.

TABLE 1
Comparison of ovarian stigma and ovulated
oocytes in treatment groups

| | PMSG/hCG | PMSG/saline | PMSG/hCG-Ant |
|---|---|---|---|
| Ovulated oocytes | 20.5 ± 2.7 | 8.33 ± 1.2 | 4.25 ± 1.4 |
| Stigma (surface) | 11.5 ± 1.1 | 7.5 ± 0.9 | 3.5 ± 1.1 |
| Stigma (serial section) | 19.5 ± 2.1 | | 9.5 ± 1.2 |

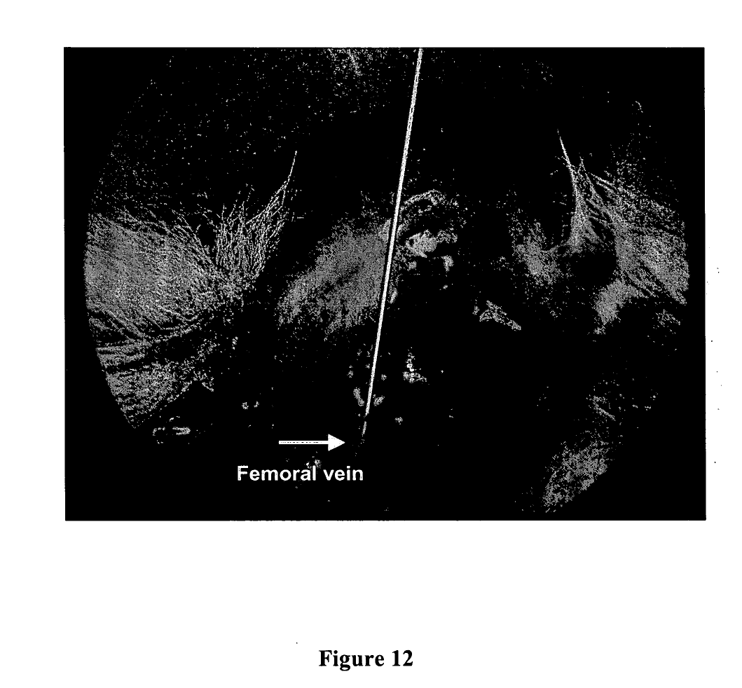

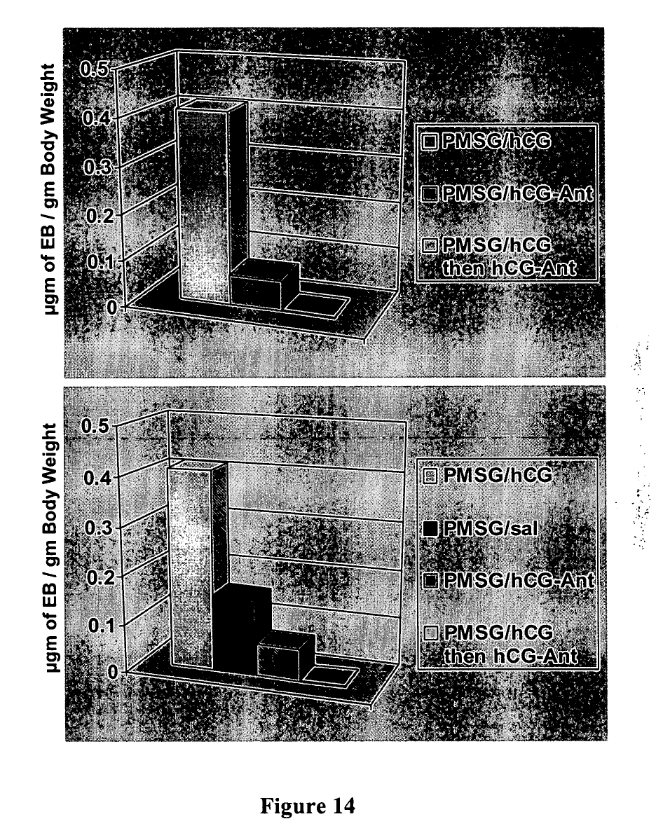

For in vivo vascular permeability studies, 18 rats were randomized into 4 groups (PMSG/hCG, PMSG/sal, PMSG/hCG-Ant, PMSG/hCG then hCG-Ant). Rats were hyperstimulated with 10 IU of PMSG for four days, followed by 30 IU of hCG, hCG-Ant, or saline on day 5. The final group also received 30 IU of hCG-Ant after hCG on day 6. Forty-eight hours after hCG, rats were evaluated for vascular permeability by injecting Evans blue (EB) dye via the femoral vein (FIG. 12) and determining dye extravasation into the peritoneal fluid. Rats treated with hCG-Ant had 85% less dye extravasation into the peritoneal fluid compared to hCG. Rats treated with hCG followed by hCG-Ant had a >99% decrease in dye extravasation compared to hCG. This reflects a dramatic decrease in vascular permeability due to hCG-Ant, especially following exposure to hCG (FIG. 14).

These results show that hCG is necessary for final oocyte maturation and represents the primary trigger for OHSS. By genetic manipulation, an hCG antagonist was constructed that effectively blocks the physiologic actions of hCG. Results demonstrate that hCG antagonist (hCG-Ant) binds to the LH receptor but does not activate it. hCG-Ant can be used to avert OHSS, while maintaining some follicular maturation and ovulation, as shown in a rat model. In superovulated rats, hCG-Ant significantly suppresses byt does not prevent ovulation completely. hCG-Ant limits vascular permeability in an OHSS model.

The hCG-Ant effectively binds to LH receptor but has significantly reduced bioactivity as compared to hCG in vitro. hCG-Ant, in vivo, reduces but does not prevent oocyte ovulation, and may be an effective treatment to block the progression of OHSS.

Additionally, the agents provided by the invention may be useful for IVF high-responders (i.e., egg donors), so that oocytes can be retrieved without IVF cycle cancellation.

### REFERENCES

1. Ganelin C 1992 Past approaches to discovering new drugs. In: Wermuth C (ed). Medicinal Chemistry for the 21 st Century. Blackwell Oxford, 3-12
2. Sun E, Cohen F E 1993 Computer-assisted drug discovery—a review. Gene 137:127-132
3. Chothia C, Lesk A M 1986 The relation between the divergence of sequence and structure in proteins. EMBO Journal 5:823-826
4. Wu H, Lustbader J W, Liu Y, Canfield R E, Hendrickson W A 1994 Structure of human chorionic gonadotropin at 2.6 A resolution from MAD analysis of the selenomethionyl protein. Structure 2:545-558
5. Lapthorn A J, Harris D C, Littlejohn A, Lustbader J W, Canfield R E, Machin K J, Morgan F J, Isaacs N W 1994 Crystal structure of human chorionic gonadotropin [see comments]. Nature 369:455-461
6. Ascoli M, Segaloff D L 1989 On the structure of the luteinizing hormone/chorionic gonadotropin receptor. [Review]. Endocrine Reviews 10:27-44
7. Segaloff D L, Ascoli M 1993 The lutropin/choriogonadotropin receptor . . . 4 years later. [Review]. Endocr Rev 14:324-347
8. Minegishi T, Igarashi S, Nakamura K, Nakamura M, Tano M, Shinozaki H, Miyamoto K, Ibuki Y 1994 Functional expression of the recombinant human FSH receptor. Journal of Endocrinology 141:369-375
9. Segaloff D L, Ascoli M 1992 The gonadotrophin receptors: insights from the cloning of their cDNAs. [Review]. Oxford Reviews of Reproductive Biology 14:141-168
10. Segaloff D L, Sprengel R, Nikolics K, Ascoli M 1990 Structure of the lutropin/choriogonadotropin receptor. [Review]. Recent Progress in Hormone Research 46:261-301
11. Lojun S, Bao S, Lei Z M, Rao C V 1997 Presence of functional luteinizing hormone/chorionic gonadotropin (hCG) receptors in human breast cell lines: implications supporting the premise that hCG protects women against breast cancer. Biology of Reproduction 57:1202-1210
12. Meduri G, Charnaux N, Loosfelt H, Jolivet A, Spyratos F, Brailly S, Milgrom E 1997 Luteinizing hormone/human chorionic gonadotropin receptors in breast cancer. Cancer Research 57:857-864
13. Parrott J A, Doraiswamy V, Kim G, Mosher R, Skinner M K 2001 Expression and actions of both the follicle stimulating hormone receptor and the luteinizing hormone receptor in normal ovarian surface epithelium and ovarian cancer. Molecular & Cellular Endocrinology 172:213-222
14. Stouffer R L, Grodin M S, Davis J R, Surwit E A 1984 Investigation of binding sites for follicle-stimulating hormone and chorionic gonadotropin in human ovarian cancers. J Clin Endocrinol Metab 59:441-446
15. Tao Y X, Bao S, Ackermann D M, Lei Z M, Rao C V 1997 Expression of luteinizing hormone/human chorionic gonadotropin receptor gene in benign prostatic hyperplasia and in prostate carcinoma in humans. Biology of Reproduction 56:67-72
16. Ben Josef E, Yang S Y, Ji T H, Bidart J M, Garde S V, Chopra D P, Porter A T, Tang D G 1999 Hormone-refractory prostate cancer cells express functional follicle-stimulating hormone receptor (FSHR). J Urol 161:970-976
17. Moyle W R, Campbell R K, Rao S N, Ayad N G, Bernard M P, Han Y, Wang Y 1995 Model of human chorionic gonadotropin and lutropin receptor interaction that explains signal transduction of the glycoprotein hormones. Journal of Biological Chemistry 270:20020-20031
18. Bernard M P, Myers R V, Moyle W R 1998 Lutropins appear to contact two independent sites in the extracellular domain of their receptors. Biochem J 335 (Pt 3):611-617
19. Ji I, Ji T H 1993 Receptor activation is distinct from hormone binding in intact lutropin-choriogonadotropin receptors and Asp397 is important for receptor activation. J Biol Chem 268:20851-20854
20. Ryu K, Lee H, Kim S, Beauchamp J, Tung C S, Isaacs N R, Ji I, Ji T H 1998 Modulation of high affinity hormone binding. Human choriogonadotropin binding to the exodomain of the receptor is influenced by exoloop 2 of the receptor. J Biol Chem 273:6285-6291
21. Ryu K, Gilchrist R L, Tung C S, Ji I, Ji T H 1998 High affinity hormone binding to the extracellular N-terminal exodomain of the follicle-stimulating hormone receptor is critically modulated by exoloop 3. J Biol Chem 273:28953-28958
22. Dufau M L 1998 The luteinizing hormone receptor. Annu Rev Physiol 60:461-496
23. Braun T, Schofield P R, Sprengel R 1991 Amino-terminal leucine-rich repeats in gonadotropin receptors determine hormone selectivity. EMBO Journal 10:1885-1890
24. Remy J J, Couture L, Pantel J, Haertle T, Rabesona H, Bozon V, Pajot-Augy E, Robert P, Troalen F, Salesse R, Bidart J M 1996 Mapping of HCG-receptor complexes. Mol Cell Endocrinol 125:79-91
25. Thomas D, Rozell T G, Liu X, Segaloff D L 1996 Mutational analyses of the extracellular domain of the full-length lutropin/choriogonadotropin receptor suggest leucine-rich repeats 1-6 are involved in hormone binding. Mol Endocrinol 10:760-768
26. Zhang R, Buczko E, Dufau M L 1996 Requirement of cysteine residues in exons 1-6 of the extracellular domain of the luteinizing hormone receptor for gonadotropin binding. Journal of Biological Chemistry 271:5755-5760
27. Song Y S, Ji I, Beauchamp J, Isaacs N R, Ji T H 2000 Hormone Interactions to Leu Rich Repeats in the Gonadotropin Receptors: II. Analysis of Leu Rich Repeat 4 of Human Luteinizing Hormone/Chorionic Gonadotropin Receptor. Journal of Biological Chemistry
28. Jeoung M, Phang T, Song Y S, Ji I, Ji T H 2000 Hormone Interactions to Leu Rich Repeats in the Gonadotropin Receptors: III. Photoaffinity labeling of Human Chorionic Gonadotropin with Receptor Leu Rich Repeat 4 Peptide. Journal of Biological Chemistry
29. Song Y S, Ji I, Beauchamp J, Isaacs N R, Ji T H 2000 Hormone Interactions to Leu Rich Repeats in the Gonadotropin Receptors: I. Analysis of Leu Rich Repeats of Human Luteinizing Hormone/Chorionic Gonadotropin Receptor and Follicle Stimulating Hormone Receptor. Journal of Biological Chemistry
30. Hong S H, Ji T H, Ji T H 1999 The beta-subunit of human choriogonadotropin interacts with the exodomain of the luteinizing hormone/choriogonadotropin receptor and changes its interaction with the alpha-subunit. Mol Endocrinol 13:1285-1294
31. Hong S, Ji I, Ji T H 1999 The alpha-subunit of human choriogonadotropin interacts with the exodomain of the luteinizing hormone/choriogonadotropin receptor. Endocrinology 140:2486-2493
32. Nakabayashi K, Kudo M, Kobilka B, Hsueh A J 2000 Activation of the luteinizing hormone receptor following substitution of ser-277 with selective hydrophobic residues in the ectodomain hinge region. Journal of Biological Chemistry 275:30264-30271
33. Kudo M, Osuga Y, Kobilka B K, Hsueh A J W 1996 Transmembrane regions V and VI of the human luteinizing hormone receptor are required for constitutive activation by a mutation in the third intracellular loop. Journal of Biological Chemistry 271:22470-22478
34. Puett D, Bhowmick N, Fernandez L M, Huang J, Wu C, Narayan P 1996 hCG-receptor binding and transmembrane signaling. Mol Cell Endocrinol 125:55-64
35. Chen W, Bahl O P 1993 High expression of the hormone binding active extracellular domain (1-294) of rat lutropin receptor in Escherichia coli. Molecular & Cellular Endocrinology 91:35-41
36. Jia X C, Oikawa M, Bo M, Tanaka T, Ny T, Boime I, Hsueh A J 1991 Expression of human luteinizing hormone (LH) receptor: interaction with LH and chorionic gonadotropin from human but not equine, rat, and ovine species. Molecular Endocrinology 91:759-768
37. Narayan P, Gray J, Puett D 1996 Expression of functional lutropin/choriogonadotropin receptor in the baculovirus system. Molecular & Cellular Endocrinology 117:95-100
38. Osuga Y, Hayashi M, Kudo M, Conti M, Kobilka B, Hsueh A J 1997 Co-expression of defective luteinizing hormone receptor fragments partially reconstitutes ligand-induced signal generation. Journal of Biological Chemistry 272:25006-25012
39. Morbeck D E, Roche P C, Keutmann H T, McCormick D J 1993 A receptor binding site identified in the region 81-95 of the beta-subunit of human luteinizing hormone (LH) and chorionic gonadotropin (hCG). Molecular & Cellular Endocrinology 97:173-181
40. Charlesworth M C, McCormick D J, Madden B, Ryan R J 1987 Inhibition of human choriotropin binding to receptor by human choriotropin alpha peptides. A comprehensive synthetic approach. Journal of Biological Chemistry 262:13409-13416
41. Roche P C, Ryan R J, McCormick D J 1992 Identification of hormone-binding regions of the luteinizing hormone/human chorionic gonadotropin receptor using synthetic peptides. Endocrinology 131:268-274
42. Phang T, Kundu G, Hong S, Ji I, Ji T H 1998 The amino-terminal region of the luteinizing hormone/choriogonadotropin receptor contacts both subunits of human choriogonadotropin. II. Photoaffinity labeling. J Biol Chem 273:13841-13847
43. Hong S, Phang T, Ji I, Ji T H 1998 The amino-terminal region of the luteinizing hormone/choriogonadotropin receptor contacts both subunits of human choriogonadotropin. I. Mutational analysis. J Biol Chem 273:13835-13840
44. Fernandez L M, Puett D 1996 Identification of amino acid residues in transmembrane helices VI and VII of the lutropin/choriogonadotropin receptor involved in signaling. Biochemistry 35:3986-3993
45. O'Connell Y, McKenna T J, Cunningham S K 1994 The effect of prolactin, human chorionic gonadotropin, insulin and insulin-like growth factor 1 on adrenal steroidogenesis in isolated guinea-pig adrenal cells. J Steroid Biochem Mol Biol 48:235-240
46. Bhalla V K, Browne E S, Sohal G S 1987 Demonstration of hCG binding sites and hCG stimulated steroidogenesis in different populations of interstitial cells. Adv Exp Med Biol 219:489-513
47. Huang C C, Wu N A, Lee M S 1993 Effects of gonadotropin on steroidogenesis by luteinized human granulosa cells. J Formos Med Assoc 92:618-622
48. Cole L A, Kardana A, Seifer D B, Bohler H C 1994 Urine hCG beta-subunit core fragment, a sensitive test for ectopic pregnancy. J Clin Endocrinol Metab 78:497-499
49. Cole L A, Kohom E I, Kim G S 1994 Detecting and monitoring trophoblastic disease. New perspectives on measuring human chorionic gonadotropin levels. J Reprod Med 39:193-200
50. Marcillac I, Troalen F, Bidart J M, Ghillani P, Ribrag V, Escudier B, Malassagne B, Droz J P, Lhomme C, Rougier P. 1992 Free human chorionic gonadotropin beta subunit in gonadal and nongonadal neoplasms. Cancer Research 52:3901-3907
51. Kornyei J L, Lei Z M, Rao C V 1993 Human myometrial smooth muscle cells are novel targets of direct regulation by human chorionic gonadotropin. Biology of Reproduction 49:1149-1157
52. Lincoln S R, Lei Z M, Rao C V, Yussman M A 1992 The expression of human chorionic gonadotropin/human luteinizing hormone receptors in ectopic human endometrial implants. Clin Endocrinol Metab 75:1140-1144
53. Lei Z M, Toth P, Rao C V, Pridham D 1993 Novel coexpression of human chorionic gonadotropin (hCG)/human luteinizing hormone receptors and their ligand hCG in human fallopian tubes. J Clin Endocrinol Metab 77:863-872
54. Hay D L, Lopata A 1988 Chorionic gonadotropin secretion by human embryos in vitro. J Clin Endocrinol Metab 67:1322-1324
55. Dimitriadou F, Phocas I, Mantzavinos T, Sarandakou A, Rizos D, Zourlas P A 1992 Discordant secretion of pregnancy specific beta 1-glycoprotein and human chorionic gonadotropin by human pre-embryos cultured in vitro. Fertility & Sterility 57:631-636
56. Iles R K, Purkis P E, Whitehead P C, Oliver R T, Leigh I, Chard T 1990 Expression of beta human chorionic gonadotrophin by non-trophoblastic non-endocrine ‘normal’ and malignant epithelial cells. British Journal of Cancer 61:663-666
57. Kumar S, Talwar G P, Biswas D K 1992 Necrosis and inhibition of growth of human lung tumor by anti-alpha-human chorionic gonadotropin antibody. J Natl Cancer Inst 84:42-47
58. Shi Q J, Lei Z M, Rao C V, Lin J 1993 Novel role of human chorionic gonadotropin in differentiation of human cytotrophoblasts. Endocrinology 132:1387-1395
59. Blithe D L, Richards R G, Skarulis M C 1991 Free alpha molecules from pregnancy stimulate secretion of prolactin from human decidual cells: a novel function for free alpha in pregnancy. Endocrinology 129:2257-2259
60. Dokras A, Sargent I L, Ross C, Gardner R L, Barlow D H 1991 The human blastocyst: morphology and human chorionic gonadotrophin secretion in vitro. Human Reproduction 6:1143-1151
61. Woodward B J, Lenton E A, Turner K, Grace W F 1994 Embryonic human chorionic gonadotropin secretion and hatching: poor correlation with cleavage rate and morphological assessment during preimplantation development in vitro. Human Reproduction 9:1909-1914
62. Dokras A, Sargent I L, Barlow D H 1993 Human blastocyst grading: an indicator of developmental potential? Human Reproduction 8:2119-2127
63. Dokras A, Sargent I L, Gardner R L, Barlow D H 1991 Human trophectoderm biopsy and secretion of chorionic gonadotrophin. Human Reproduction 6:1453-1459
64. Lopata A, Hay D L 1989 The potential of early human embryos to form blastocysts, hatch from their zona and secrete HCG in culture. Human Reproduction 4:87-94
65. Bonnamy P J, Benhaim A, Leymarie P 1993 Uterine luteinizing hormone/human chorionic gonadotropin-binding sites in the early pregnant rat uterus: evidence for total occupancy in the periimplantation period. Endocrinology 132:1240-1246
66. Tapanainen J S, Tilly J L, Vihko K K, Hsueh A J 1993 Hormonal control of apoptotic cell death in the testis: gonadotropins and androgens as testicular cell survival factors. Mol Endocrinol 7:643-650
67. Gompel A, Sabourin J C, Martin A, Yaneva H, Audouin J, Decroix Y, Poitout P 1994 Bcl-2 expression in normal endometrium during the menstrual cycle. Am J Pathol 144:1195-1202
68. Lobel L, Pollak S, Wang S, Chaney M, Lustbader J W 1999 Expression and characterization of recombinant beta-subunit hCG homodimer. Endocrine 10:261-270
69. Moyle W R, Myers R V, Wang Y, Han Y, Lin W, Kelley G L, Ehrlich P H, Rao S N, Bernard M P 1998 Functional homodimeric glycoprotein hormones: implications for hormone action and evolution. Chemistry & Biology 5:241-254
70. Ikura M, Kay L E, Bax A 1990 A novel approach for sequential assignment of 1H, 13C, and 15N spectra of proteins: heteronuclear triple-resonance three-dimensional NMR spectroscopy. Application to calmodulin. Biochemistry 29:4659-4667
71. Lobel L I, Rausch P, Trakht I, Pollak S, Lustbader J W 1997 Filamentous phage displaying the extracellular domain of the hLH/CG receptor bind hCG specifically. Endocrinology 138:1232-1239
72. Prinz W A, Aslund F, Holmgren A, Beckwith J 1997 The role of the thioredoxin and glutaredoxin pathways in reducing protein disulfide bonds in the Escherichia coli cytoplasm. Journal of Biological Chemistry 272:15661-15667
73. Derman A I, Prinz W A, Belin D, Beckwith J 1993 Mutations that allow disulfide bond formation in the cytoplasm of Escherichia coli. Science 262:1744-1747
74. Lobel L I, Pollak S, Klein J, Lustbader J W 2001 High-Level Bacterial Expression of a Natively Folded, Soluble Extracellular Domain Fusion Protein of the Human Luteinizing Hormone/Chorionic Gonadotropin Receptor in the Cytoplasm of Escherichia coli. Endocrine 14:
75. Dufau M L, Kusuda S 1987 Purification and characterization of ovarian LH/hCG and prolactin receptors. Journal of Receptor Research 7:167-193
76. Lustbader J W, Yarmush D L, Birken S, Puett D, Canfield R E 1993 The application of chemical studies of human chorionic gonadotropin to visualize its three-dimensional structure. [Review]. Endocrine Reviews 14:291-311
77. O'Connor J F, Birken S, Lustbader J W, Krichevsky A, Chen Y, Canfield R E 1994 Recent advances in the chemistry and immunochemistry of human chorionic gonadotropin: impact on clinical measurements. [Review]. Endocrine Reviews 15:650-683
78. McPherson A, Koszelak S, Axelrod H, Day J, Williams R, Robinson L, McGrath M, Cascio D 1986 An experiment regarding crystallization of soluble proteins in the presence of beta-octyl glucoside. Journal of Biological Chemistry 261:1969-1975
79. Yurkova E V, Demin V V, Abdulaev N G 1990 Crystallization of membrane proteins: bovine rhodopsin. Biomed Sci 1:585-590
80. Hirsch A, Wacker T, Weckesser J, Diederichs K, Welte W 1995 Purification, characterization, crystallization, and preliminary X-ray results from Paracoccus denitrificans porin. Proteins 23:282-284
81. Arockiasamy A, Krishnaswamy S 1999 Crystallization of the immunodominant outer membrane protein OmpC; the first protein crystals from Salmonella typhi, a human pathogen. FEBS Lett 453:380-382
82. Davies A, Schertler G F, Gowen B E, Saibil H R 1996 Projection structure of an invertebrate rhodopsin. J Struct Biol 117:36-44
83. Lustbader J W, Wu H, Birken S, Pollak S, Gawinowicz Kolks M A, Pound A M, Austen D, Hendrickson W A, Canfield R E 1995 The expression, characterization, and crystallization of wild-type and selenomethionyl human chorionic gonadotropin. Endocrinology 136:640-650
84. Powell A J, Read J A, Banfield M J, Gunn-Moore F, Yan S D, Lustbader J, Stem A R, Stem D M, Brady R L 2000 Recognition of structurally diverse substrates by type II 3-hydroxyacyl-CoA dehydrogenase (HADH II)/Amyloid-beta binding alcohol dehydrogenase (ABAD). Journal of Molecular Biology 303:311-327
85. Zeng H, Phang T, Ji I, Ji T H 2000 The role of the hinge region in the luteinizing hormone receptor in hormone interaction and signal generation. Journal of Biological Chemistry
86. Graf K M, Dias J A, Griswold M D 1997 Decreased spermatogenesis as the result of an induced autoimmune reaction directed against the gonadotropin receptors in male rats. J Androl 18:174-185
87. Remy J J, Bozon V, Couture L, Goxe B, Salesse R, Garnier J 1993 Suppression of fertility in male mice by immunization against LH receptor. J Reprod Immunol 25:63-79
88. Laemmli U K 1970 Cleavage of structural proteins during the assembly of the head of bacteriophage T4. Nature 70:680-685
89. Laemmli U K, Favre M 1973 Maturation of the head of bacteriophage T4.1. DNA packaging events. Journal of Molecular Biology 73:575-599
90. Towbin H, Staehelin T, Gordon J 1979 Electrophoretic transfer of proteins from polyacrylamide gels to nitrocellulose sheets: procedure and some applications. Proceedings of the National Academy of Sciences of the United States of America 76:4350-4354
91. Burnette W N 1981 “Western blotting”: electrophoretic transfer of proteins from sodium dodecyl sulfate—polyacrylamide gels to unmodified nitrocellulose and radiographic detection with antibody and radioiodinated protein A. Analytical Biochemistry 112:195-203
92. Gray J W, Dolbeare F, Pallavicini M G, Beisker W, Waldman F 1986 Cell cycle analysis using flow cytometry. Int J Radiat Biol Relat Stud Phys Chem Med 49:237-255
93. Lustbader J W, Birken S, Pileggi N F, Kolks M A, Pollak S, Cuff M E, Yang W, Hendrickson W A, Canfield R E 1989 Crystallization and characterization of human chorionic gonadotropin in chemically deglycosylated and enzymatically desialylated states. Biochemistry 28:9239-9243
94. Remy J J, Nespoulous C, Grosclaude J, Grebert D, Couture L, Pajot E, Salesse R 2001 Purification and Structural Analysis of a Soluble Human Chorionogonadotropin Hormone-Receptor Complex. Journal of Biological Chemistry 276:1681-1687
95. Scorer C A, Buckholz R G, Clare J J, Romanos M A 1993 The intracellular production and secretion of HIV-1 envelope protein in the methylotrophic yeast Pichia pastoris. Gene 136:111-119
96. Weiss H M, Haase W, Michel H, Reilander H 1995 Expression of functional mouse 5-HT5A serotonin receptor in the methylotrophic yeast Pichia pastoris: pharmacological characterization and localization. FEBS Lett 377:451-456
97. Gachhui R, Presta A, Bentley D F, Abu-Soud H M, McArthur R, Brudvig G, Ghosh D K, Stuehr D J 1996 Characterization of the reductase domain of rat neuronal nitric oxide synthase generated in the methylotrophic yeast Pichia pastoris. Calmodulin response is complete within the reductase domain itself. Journal of Biological Chemistry 271:20594-20602
98. Trimble R B, Atkinson P H, Tschopp J F, Townsend R R, Maley F 1991 Structure of oligosaccharides on Saccharomyces SUC2 invertase secreted by the methylotrophic yeast Pichia pastoris. Journal of Biological Chemistry 266:22807-22817
99. Kwong P D, Wyatt R, Desjardins E, Robinson J, Culp J S, Hellmig B D, Sweet R W, Sodroski J, Hendrickson W A 1999 Probability analysis of variational crystallization and its application to gp120, the exterior envelope glycoprotein of type I human immunodeficiency virus (HIV-1). Journal of Biological Chemistry 274:4115-4123
100. Liu X, DePasquale J A, Griswold M D, Dias J A 1994 Accessibility of rat and human follitropin receptor primary sequence (R265-S296) in situ. Endocrinology 135:682-691
101. Ji I, Ji T H 1995 Differential roles of exoloop 1 of the human follicle-stimulating hormone receptor in hormone binding and receptor activation. Journal of Biological Chemistry 270:15970-15973
102. Moyle W R, Ehrlich P H, Canfield R E 1982 Use of monoclonal antibodies to subunits of human chorionic gonadotropin to examine the orientation of the hormone in its complex with receptor. Proceedings of the National Academy of Sciences of the United States of America 79:2245-2249
103. Hendrickson W A, Horton J R, LeMaster D M 1990 Selenomethionyl proteins produced for analysis by multiwavelength anomalous diffraction (MAD): a vehicle for direct determination of three-dimensional structure. EMBO Journal 9:1665-1672
104. Leahy D J, Erickson H P, Aukhil I, Joshi P, Hendrickson W A 1994 Crystallization of a fragment of human fibronectin: introduction of methionine by site-directed mutagenesis to allow phasing via selenomethionine. Proteins 19:48-54
105. Carter C W, Carter C W 1979 Protein crystallization using incomplete factorial experiments. Journal of Biological Chemistry 254:12219-12223
106. Gouaux E, Hobaugh M, Song L 1997 alpha-Hemolysin, gamma-hemolysin, and leukocidin from Staphylococcus aureus: distant in sequence but similar in structure. Protein Science 6:2631-2635
107. Song L, Hobaugh M R, Shustak C, Cheley S, Bayley H, Gouaux J E 1996 Structure of staphylococcal alpha-hemolysin, a heptameric transmembrane pore. Science 274:1859-1866
108. Gouaux J E, Braha O, Hobaugh M R, Song L, Cheley S, Shustak C, Bayley H 1994 Subunit stoichiometry of staphylococcal alpha-hemolysin in crystals and on membranes: a heptameric transmembrane pore. Proc Natl Acad Sci USA 91:12828-12831
109. Crystallization of membrane proteins 1991 CRC Press,
110. Ostermeier C, Iwata S, Ludwig B, Michel H 1995 Fv fragment-mediated crystallization of the membrane protein bacterial cytochrome c oxidase. Nature Structural Biology 2:842-846
111. Pollak S, Halpine S, Chait B T, Birken S 1990 High resolution high performance liquid chromatography fingerprinting of purified human chorionic gonadotropin demonstrates that oxidation is a cause of hormone heterogeneity. Endocrinology 126:199-208
112. Ji I, Ji T H 1980 Macromolecular photoaffinity labeling of the lutropin receptor on granulosa cells. Proc Natl Acad Sci USA 77:7167-7170
113. Ji T H, Kiehm D J, Middaugh C R 1980 Presence of spectrin tetramer on the erythrocyte membrane. Journal of Biological Chemistry 255:2990-2993
114. Iles R K, Chard T 1989 Immunochemical analysis of the human chorionic gonadotrophin-like material secreted by ‘normal’ and neoplastic urothelial cells. J Mol Endocrinol 2:107-112
115. Moyle W R, Campbell R K, Myers R V, Bernard M P, Han Y, Wang X 1994 Co-evolution of ligand-receptor pairs. Nature 368:251-255
116. Campbell R K, Dean-Emig D M, Moyle W R 1991 Conversion of human choriogonadotropin into a follitropin by protein engineering. Proc Natl Acad Sci USA 88:760-764
117. Hansen M B, Nielsen S E, Berg K 1989 Re-examination and further development of a precise and rapid dye method for measuring cell growth/cell kill. Journal of Immunological Methods 119:203-210
118. Mosmann T 1983 Rapid colorimetric assay for cellular growth and survival: application to proliferation and cytotoxicity assays. Journal of Immunological Methods 65:55-63
119. Dolbeare F, Kuo W L, Beisker W, Vanderlaan M, Gray J W 1990 Using monoclonal antibodies in bromodeoxyuridine-DNA analysis. Methods Cell Biol 33:207-216
120. McCarthy N J, Smith C A, Williams G T 1992 Apoptosis in the development of the immune system: growth factors, clonal selection and bcl-2. Cancer Metastasis Rev 11:157-178
121. Carson W E, Haldar S, Baiocchi R A, Croce C M, Caligiuri M A 1994 The c-kit ligand suppresses apoptosis of human natural killer cells through the upregulation of bcl-2. Proc Natl Acad Sci USA 91:7553-7557
122. Sato N, Hotta K, Waguri S, Nitatori T, Tohyama K, Tsujimoto Y, Uchiyama Y 1994 Neuronal differentiation of PC 12 cells as a result of prevention of cell death by bcl-2. J Neurobiol 25:1227-1234
123. Sorkin A, Waters C M 1993 Endocytosis of growth factor receptors. Bioessays 15:375-382
124. Cleveland D W, Fischer S G, Kirschner M W, Laemmli U K 1977 Peptide mapping by limited proteolysis in sodium dodecyl sulfate and analysis by gel electrophoresis. Journal of Biological Chemistry 252:1102-1106
125. Petaja-Repo U E, Merz W E, Rajaniemi H J 1993 Significance of the carbohydrate moiety of the rat ovarian luteinizing-hormone/chorionic-gonadotropin receptor for ligand-binding specificity and signal transduction. Biochemical Journal 292 (Pt 3):839-844
126. Licht P, Cao H, Zuo J, Lei Z M, Rao V, Merz W E, Day T G 1994 Lack of self-regulation of human chorionic gonadotropin biosynthesis in human choriocarcinoma cells. J Clin Endocrinol Metab 78:1188-1194
127. Edwards R G 1994 Implantation, interception and contraception. Human Reproduction 9:985-995
128. Bongso A, Fong C Y, Ng S C, Ratnam S 1994 Human embryonic behavior in a sequential human oviduct-endometrial coculture system. Fertility & Sterility 61:976-978
129. Woodward B J, Lenton E A, Turner K 1993 Human chorionic gonadotrophin: embryonic secretion is a time-dependent phenomenon. Human Reproduction 8:1463-1468
130. Gunn L K, Homa S T, Searle M J, Chard T 1994 Lack of evidence for the production of interferon-alpha-like species by the cultured human pre-embryo. Human Reproduction 9:1522-1527
131. weitlauf H M 1988 In: Knobil E, Neill J (eds). The Physiology of Reproduction. Raven, New York: 231-263
132. Ilesanmi A O, Hawkins D A, Lessey B A 1993 Immunohistochemical markers of uterine receptivity in the human endometrium. Microsc Res Tech 25:208-222
133. Horvat B 1993 Galactose-binding lectins as markers of pregnancy-related glycoproteins. Histochemistry 99:95-101
134. Mathialagan N, Roberts R M 1994 A role for cytokines in early pregnancy. Indian J Physiol Pharmacol 38:153-162
135. Das S K, Wang X N, Paria B C, Damm D, Abraham J A, Klagsbrun M, Andrews G K, Dey S K 1994 Heparin-binding EGF-like growth factor gene is induced in the mouse uterus temporally by the blastocyst solely at the site of its apposition: a possible ligand for interaction with blastocyst EGF-receptor in implantation. Development 120:1071-1083
136. Nader S, Berkowitz A S, Ochs D, Held B, Winkel C A 1988 Luteal-phase support in stimulated cycles in an in vitro fertilization/embryo transfer program: progesterone versus human chorionic gonadotropin. J In Vitro Fert Embryo Transf 5:81-84
137. Hustin J, Philippe E, Teisner B, Grudzinskas J G 1994 Immunohistochemical localization of two endometrial proteins in the early days of human pregnancy. Placenta 15:701-708
138. Chatzaki E, Gallagher C J, Iles R K, Ind T E, Nouri A M, Bax C M, Grudzinskas J G 1994 Characterisation of the differential expression of marker antigens by normal and malignant endometrial epithelium. British Journal of Cancer 69:1010-1014
139. Mellor S J, Thomas E J 1994 The actions of estradiol and epidermal growth factor in endometrial and endometriotic stroma in vitro. Fertility & Sterility 62:507-513
140. Ren S G, Braunstein G D 1990 Progesterone and human chorionic gonadotropin do not stimulate placental proteins 12 and 14 or prolactin production by human decidual tissue in vitro. J Clin Endocrinol Metab 70:983-989
141. Bonfrer J M, Baan A W, Jansen E, Lentfer D, Kenemans P 1994 Technical evaluation of three second generation CA 125 assays. Eur J Clin Chem Clin Biochem 32:201-207

What is claimed is:

1. A human chorionic gonadotropin (hCG) antagonist comprising deglycosylated hCG, wherein the deglycosylated hCG is recombinantly produced.

2. The hCG antagonist of claim 1, wherein the deglycosylated hCG comprises a reduced number of carbohydrate residues relative to naturally occurring hCG.

3. The hCG antagonist of claim 1, wherein the deglycosylated hCG comprises less N-linked carbohydrates than naturally occurring hCG.

4. The hCG antagonist of claim 1, wherein the deglycosylated hCG has at least about 50% to at least about 90% less carbohydrates than naturally occurring hCG.

5. The hCG antagonist of claim 1, wherein the deglycosylated hCG is expressed in a host cell that is glycosylation-deficient.

6. The hCG antagonist of claim 5, wherein the host cell lacks a N-acetylglucosaminyl transferase enzyme.

7. The hCG antagonist of claim 5, wherein the host cell is Chinese hamster ovary cell.

8. A human chorionic gonadotropin (hCG) antagonist comprising deglycosylated hCG, wherein the deglycosylated hCG comprises at least one hCG-α subunit and at least one hCG-β subunit fused together.

9. The hCG antagonist of claim 8, wherein the deglycosylated hCG comprises a reduced number of carbohydrate residues relative to naturally occurring hCG.

10. The hCG antagonist of claim 8, wherein the at least one hCG-α subunit has less carbohydrates than a naturally occurring hCG-α subunit.

11. The hCG antagonist of claim 8, wherein the at least one hCG-β subunit has less carbohydrates than a naturally occurring hCG-β subunit.

12. An hCG antagonist comprising a mutated hCG encoded by the nucleic acid sequence of SEQ ID NO:1.

13. A composition comprising the human chorionic gonadotropin (hCG) antagonist of claim 1 and a pharmaceutically acceptable carrier.

14. A composition comprising the human chorionic gonadotropin (hCG) antagonist of claim 8 and a pharmaceutically acceptable carrier.

15. A method for making a human chorionic gonadotropin (hCG) antagonist comprising transforming a host cell with the vector comprising a nucleic acid encoding hCG, wherein the host cell is glycosylation-deficient, and culturing the host cell under conditions sufficient to obtain expression of hCG, wherein the expressed hCG is deglycosylated.

16. The method of claim 15, wherein the deglycosylated hCG comprises a reduced number of carbohydrate residues relative to naturally occurring hCG.

17. The method of claim 15, wherein the deglycosylated hCG comprises less N-linked carbohydrates than naturally occurring hCG.

18. The method of claim 15, wherein the deglycosylated hCG has at least about 50% to at least about 90% less carbohydrates than naturally occurring hCG.

19. The method of claim 15, wherein the deglycosylated hCG is encoded by a nucleic acid having the sequence of SEQ ID NO:1.

20. A host cell expressing deglycosylated hCG, wherein the host cell is glycosylation-deficient.

21. A method for inhibiting the activity of naturally-occurring human chorionic gonadotropin (hCG) via competitive inhibition comprising administering to a subject in need thereof an hCG antagonist comprising deglycosylated hCG in a hCG-antagonizing amount.

22. The method of claim 21, wherein the deglycosylated hCG is recombinantly expressed.

23. The method of claim 22, wherein the deglycosylated hCG is recombinantly expressed in a glycosylation-deficient host cell.

24. The method of claim 23, wherein the host cell is Chinese hamster ovary cells.

25. The method of claim 21, wherein the hCG antagonist is administered orally, intravenously, intramuscularly, or subcutaneously.

26. The method of claim 21, wherein the hCG antagonist is administered in combination with a hormone or a chemotherapeutic agent.

27. The method of claim 26, wherein the hCG antagonist is administered both before and after the hormone.

28. The method of claim 21, wherein the hCG antagonist is administered in an amount from about 3,000 IU to about 10,000 IU.

29. The method of claim 21, wherein the subject receiving the hCG antagonist suffers from ovarian hyperstimulation syndrome, gestational trophoblastic disease, proliferation of circulating hCG, proliferation of hCG-α, proliferation of hCG-β, tumors secreting hCG, or tumors responsive to hCG or cells with hCG receptors.

30. A method for treating ovarian hyperstimulation syndrome in a subject comprising administering to the subject an effective amount of the human chorionic gonadotropin (hCG) antagonist comprising deglycosylated hCG.

31. The method of claim 30, wherein the deglycosylated hCG is recombinantly expressed.

32. The method of claim 31, wherein the deglycosylated hCG is recombinantly expressed in a glycosylation-deficient host cell.

33. The method of claim 32, wherein the host cell is Chinese hamster ovary cells.

34. The method of claim 30, wherein the hCG antagonist is administered orally, intravenously, intramuscularly, or subcutaneously.

35. The method of claim 30, wherein the hCG antagonist is administered in combination with a hormone or a chemotherapeutic agent.

36. The method of claim 35, wherein the hCG antagonist is administered both before and after the hormone.

37. The method of claim 30, wherein the hCG antagonist is administered in an amount from about 3,000 IU to about 10,000 IU.

38. A method for treating a subject suffering from ovarian hyperstimulation syndrome comprising administering to the subject in need thereof a human chorionic gonadotropin (hCG) antagonist comprising deglycosylated hCG in a hCG-antagonizing amount.

39. The method of claim 38, wherein the subject is receiving hCG therapy

40. A method for treating a subject suffering from gestational trophoblastic disease from increased circulation of human chorionic gonadotropin (hCG) comprising administering to the subject an effective amount of a hCG antagonist comprising deglycosylated hCG.

41. The method of claim 40, wherein the deglycosylated hCG is recombinantly expressed.

42. The method of claim 41, wherein the deglycosylated hCG is recombinantly expressed in a glycosylation-deficient host cell.

43. The method of claim 40, wherein the hCG antagonist is administered orally, intravenously, intramuscularly, or subcutaneously.

44. The method of claim 40, wherein the hCG antagonist is administered in combination with a hormone or a chemotherapeutic agent.

45. The method of claim 44, wherein the hCG antagonist is administered both before and after the hormone.

46. The method of claim 40, wherein the hCG antagonist is administered in an amount from about 3,000 IU to about 10,000 IU.

47. A method for treating a subject suffering from gestational trophoblastic disease from hCG-α, hCG-β, or both, comprising administering to the subject an effective amount of a human chorionic gonadotropin (hCG) antagonist comprising deglycosylated hCG.

48. The method of claim 47, wherein the deglycosylated hCG is recombinantly expressed.

49. The method of claim 48, wherein the deglycosylated hCG is recombinantly expressed in a glycosylation-deficient host cell.

50. The method of claim 49, wherein the host cell is Chinese hamster ovary cells.

51. The method of claim 47, wherein the hCG antagonist is administered orally, intravenously, intramuscularly, or subcutaneously.

52. The method of claim 47, wherein the hCG antagonist is administered in combination with a hormone or a chemotherapeutic agent.

53. The method of claim 52, wherein the hCG antagonist is administered both before and after the hormone.

54. The method of claim 47, wherein the hCG antagonist is administered in an amount from about 3,000 IU to about 10,000 IU.

55. A method for treating a subject suffering from a human chorionic gonadotropin (hCG)-associated tumor, which comprises administering to the subject an effective amount of a human chorionic gonadotropin (hCG) antagonist comprising deglycosylated hCG.

56. The method of claim 55, wherein the hCG antagonist is administered orally, intravenously, intramuscularly, or subcutaneously.

57. The method of claim 55, wherein the subject is suffering from a trophoblastic tumor of placental or germ cell origin.

58. The method of claim 55, wherein the subject is suffering from a relapsing choriocarcinoma or a testicular germ cell tumor.

59. The method of claim 55, wherein the subject is suffering from a biliary or a pancreatic cancer.

60. The method of claim 55, wherein the subject is suffering from lung carcinoma or bladder carcinoma.

61. The hCG antagonist of claim 15, wherein the host cell is a bacterial cell, yeast cell, or insect cell.

62. The hCG antagonist of claim 1, wherein the hCG is deglycosylated by neuraminidase treatment.

63. The hCG antagonist of claim 1, wherein the hCG is deglycosylated by hydrogen fluoride treatment.

64. A human chorionic gonadotropin (hCG) antagonist comprising deglycosylated hCG, wherein the deglycosylated hCG comprises at least one hCG-α subunit and at least one hCG-β subunit non-covalently bound together.

65. A method of inhibiting growth of human chorionic gonadotropin (hCG)-associated tumor cells comprising administering to a subject in need thereof an effective amount of a human chorionic gonadotropin (hCG) comprising deglycosylated hCG.

66. The method of claim 65, wherein the hCG-associated tumor cell is a lung carcinoma tumor cell or a bladder carcinoma tumor cell.

67. The method of claim 65, wherein the hCG antagonist acts by inhibiting the binding of naturally occurring hCG to the hCG receptor via competitive inhibition.

68. An hCG having one, two, or three of the following N-linked glycosylation mutated, so as to reduce glycosylation of the hCG: asparagine at position 13, asparagine at position 30, or asparagine at position 52.

69. A human chorionic gonadotropin antagonist comprising (i) a polypeptide having an amino acid sequence of an α subunit of hCG (SEQ ID NO:2) except that the asparagine at position 52 is mutated; and (ii) a polypeptide having an amino acid sequence of a β subunit of hCG (SEQ ID NO:3) except that the asparagine at position 13 and the asparagine at position 30 are mutated.

70. The antagonist of claim 69, wherein the α and β subunits are fused together.

71. The antagonist of claim 70, wherein the fusion is via a peptide linker.


SEQUENCE LISTING (3 sequences)

SEQ ID NO: 1 - DNA, 825 bp, Homo sapiens
atgggaagcttcgtggatccagacaaggcaggggacgcaccaaggatggagatgttccaggggctgctgctgttgctgctgctgagcatgggcgggacatgggcatccaaggagccgcttcggccacggtgccgccccatcaaagccaccctggctgtggagaaggagggctgccccgtgtgcatcaccgtcaagaccaccatctgtgccggctactgccccaccatgacccgcgtgctgcagggggtcctgccggccctgcctcaggtggtgtgcaactaccgcgatgtgcgcttcgagtccatccggctccctggctgcccgcgcggcgtgaaccccgtggtctcctacgccgtggctctcagctgtcaatgtgcactctgccgccgcagcaccactgactgcgggggtcccaaggaccaccccttgacctgtgatgacccccgcttccaggactcctcttcctcaaaggcccctccccccagccttccaagcccatcccgactcccggggccctcggacaccccgatcctcccacaagaattcgctcctgatgtgcaggattgcccagaatgcacgctacaggaaaacccattcttctcccagccgggtgccccaatacttcagtgcatgggctgctgcttctctagagcatatcccactccactaaggtccaagaagacgatgttggtccaaaagaaagtcacctcagagtccacttgctgtgtagctaaatcatataacagggtcacagtaatggggggtttcaaagtggagaaccacacggcgtgccactgcagtacttgttattatcacaaatcttaa

SEQ ID NO: 2 - protein, 92 aa, Homo sapiens
APDTQDCPECTLQENPFFSQPGAPILQCMGCCFSRAYPTPLRSKKTMLVQKNVTSESTCCVAKSYNRVTVMGGFKVENHTACHCSTCYYHKS

SEQ ID NO: 3 - protein, 145 aa, Homo sapiens
SKEPLRPRCRPINATLAVEKEGCPVCITVNTTICAGYCPTMTRVLQGVLPALPQVVCNYRDVRFESIRLPGCPRGVNPVVSYAVALSCQCALCRRSTTDCGGPKDHPLTCDDPRFQDSSSSKAPPPSLPSPSRLPGPSDTPILPQ